(19) United States
(12) Patent Application Publication
(10) Pub. No.: US 2013/0129635 A1
(43) Pub. Date: May 23, 2013

(54) POLYMERIZED SHELL LIPID MICROBUBBLES AND USES THEREOF

(71) Applicants: Jon Nagy, Bozeman, MT (US); Joyce Wong, Chestnut Hill, MA (US); Adam Luce, Boston, MA (US); Ragnhild Whitaker, Tromsoe (NO); Robin Cleveland, Cuddington (GB); Yoonjee Park, Boston, MA (US)

(72) Inventors: Jon Nagy, Bozeman, MT (US); Joyce Wong, Chestnut Hill, MA (US); Adam Luce, Boston, MA (US); Ragnhild Whitaker, Tromsoe (NO); Robin Cleveland, Cuddington (GB); Yoonjee Park, Boston, MA (US)

(73) Assignee: Wilson Sonsini Goodrch & Rosati, Palo Alto, CA (US)

(21) Appl. No.: 13/699,298

(22) PCT Filed: May 24, 2011
(86) PCT No.: PCT/US11/37801

§ 371 Date: Feb. 1, 2013

Related U.S. Application Data
(60) Provisional application No. 61/347,524, filed on May 24, 2010.

(51) Int. Cl. A61K 49/22

(52) U.S. Cl. CPC A61K 49/223
USPC 424/9.52; 424/497; 514/759; 264/4.7

(57) ABSTRACT

The present invention relates to the fabrication and use of microbubbles. In some embodiments, the invention provides for the fabrication and use of polymerized shell lipid microbubbles (PSMs). The microbubbles of the invention can be used, for example, for diagnosis and treatment of a condition.

### CROSS-REFERENCE

This application claims the benefit of U.S. Provisional Application No. 61/347,524, filed May 24, 2010, which application is incorporated herein by reference.

### BACKGROUND OF INVENTION

Current ultrasound contrast agents (USCA) vary in composition from simple gas bubbles to albumin-coated bubbles to synthetic polymer bubbles. These vast differences in shell composition result in vastly differing properties, affecting key properties such as ultrasound response and in vivo circulation time. Likewise, size and size distribution exert effects over these properties. Clinically available ultrasound contrast agents are limited to polydisperse, non-targeted microbubbles, which result in non-specific highlighting of the vasculature. Targeted contrast ultrasound offers the potential to target specific molecular markers in the vasculature, revealing information about molecular makeup in addition to structure. Information about molecular makeup is crucial in many diagnostic applications, such as inflammation in atherosclerosis and angiogenesis in cancer. Additionally, by utilizing microbubbles as drug/gene delivery vehicles, drugs can be targeted to specific molecular markers and induced to release upon increased ultrasound stimulation, offering a means of image-guided drug delivery, or theranostics.

### SUMMARY OF THE INVENTION

In one aspect the invention provides a microbubble. In another aspect the invention provides kits and methods of making a using the microbubble.

In some embodiments, the invention provides a monodisperse polymerized shell lipid microbubbles (PSMs) comprising polymerizable lipids, methods of making and using this polymerized shell microbubbles in ultrasound-based diagnostic and therapeutic technologies. For example, these polymerized shell lipid microbubbles can be used in ultrasound imaging and ultrasound-induced drug delivery.

In another embodiment, the present invention provides for a microbubble comprising a polymerized lipid shell and a gas, where the gas is encased with the shell. In some embodiments, the invention provides a microbubble comprising a polymerized lipid shell and a gas, where the gas is encased within the shell, and where the polymerized lipid shell comprises at least about 5% polymerizable lipid.

In another embodiment, the present invention provides for a collection of microbubbles comprising gas-filled polymerized shell lipid microbubbles, where the microbubbles in the collection are monodispersed and are within a micrometer size range. In one embodiment, the microbubbles of the collection have all the characteristics of a microbubble described herein.

In some embodiments, the invention provides methods treating an individual comprising administering a microbubble to an individual in need of thereof, the microbubble comprising a polymerized lipid shell, a gas, a targeting agent and a therapeutic agent, where the gas is encased within the shell, and where the polymerized lipid shell comprises at least about 5% polymerizable lipid.

In one embodiment, the gas of the microbubble is a heavy gas. In one embodiment, the heavy gas is a perfluorocarbon. In another embodiment, the gas of the microbubble is a mixture of at least two perfluorocarbons. In one embodiment, the perfluorocarbon is decafluorobutane.

In one embodiment, the polymerized lipid shell of the microbubble comprises at least one polymerizable lipid and at least one non-polymerizable lipid and has a percentage of about 5-50% polymerizable lipid. In one embodiment, the at least one non-polymerizable lipid is selected from the group consisting of L-α-phosphatidylcholine, PE-PEG2000 (1,2-distearoyl-sn-glycero-3-phosphoethanolatnine-N-[methoxy(polyethylene glycol)-2000 or PE-PEG2000-biotin. In one embodiment, the polymerized lipid shell comprises a percentage of PEGylated lipid between about 1-20%. In one embodiment, the lipid is non-polymerizable and PEGylated. In one embodiment, the lipid is polymerizable and PEGylated. In one embodiment, the at least one polymerizable lipid is a diacetylenic lipid.

In one embodiment, the microbubble is UV treated for about 2-5 minutes after fabrication to polymerize the lipid shell. In one embodiment, the microbubble is UV treated for about 30 minutes after fabrication to polymerize the lipid shell. In one embodiment, the microbubble has an absorbance at a wavelength between about 400-580 nm.

In one embodiment, the microbubble has a micrometer size range is about 2-5 μm. In one embodiment, the collection of microbubbles is monodispersed, and the monodisperity is about 10% to about 20% of an average size of the microbubbles in the collection. In one embodiment, the average size of the microbubbles in the collection is between about 2-5 μm.

In some embodiments, the microbubbles are prepared by a microfluidic flow focusing device.

In some embodiments, the microbubble comprises a targeting agent. In some embodiments, the microbubble is conjugated with a ligand and the conjugation is by way of the tethering the ligand to the lipid shell. In some embodiment, the microbubbles comprise a targeting agent. Examples of targeting agents include, but are not limited to, drug, a chemical, antibodies, ligands, proteins, peptides, carbohydrates, vitamins, nucleic acids, an agent, any entity within the shell or a combinations thereof. In some embodiments, the targeting agent is specific to a cell surface molecule. In some embodiments, the therapeutic agent within the shell is delivered to a target location by way of the microbubble.

In some embodiments, the microbubble is an ultrasound contrast agent further comprising an acceptable carrier for administration to an individual. In some embodiments, the microbubble retains at least 90% of its signal after two seconds of ultrasound insonation.

In some embodiments, the microbubble has a circulation half life of about 3 to about 4 hours.

In some embodiment, the present invention provides for a method of making a microbubble of claim 1 comprising: (a) microfluidic flow focusing a mixture of polymerizable lipid and standard non-polymerizable lipid and a gas through an aperture to form micrometer microbubbles and (b) UV treating the microbubbles for at least 2 minutes to polymerize the polymerizable lipid. In some embodiments, the polymerization is by diacetylene polymerization. In some embodiments, the microbubbles are treated with UV for at least 30 minutes to polymerize the polymerizable lipid. In some embodiments, the microbubbles are treated with UV for at least 1 hour to polymerize the polymerizable lipid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Schematic of the single emulsion technique.

FIG. 2. Overarching design goals

FIG. 3. AUTOCAD design focusing on channels near the orifice.

FIG. 4. Flowchart for fabrication of a microfluidic flow focusing device.

FIG. 5. Setup for the ultrasound visualization of microbubbles.

FIG. 6. Chemical structure of Nile red.

FIG. 7A-D. Microbubble production in a flow focusing device.

FIG. 8A Microbubbles in solution exiting from the output port and floating upward following production in a microfluidic device. FIG. 8B Microbubbles floating in solution after exiting microfluidic device, prior to collection for analysis.

FIG. 9. Microbubbles following production in the microfluidic device.

FIG. 10. Dynamic light scattering data showing two populations of particles.

FIG. 11. A monodisperse microbubble population.

FIG. 12. Absorbance spectra demonstrating microbubble polymerization.

FIG. 13. Fluorescence of microbubble solutions.

FIG. 14. Effect of lipid formulations on microbubble dissolution.

FIG. 15. Results of ultrasound insonation of a PAAM-gel containing microbubbles.

FIG. 16. Time study of ultrasound contrast dissipation over time.

FIG. 17A-D. Results Nile red encapsulation studies. Scale bar is for all images. FIG. 17A is a brightfield image of lipid microbubbles with no dye encapsulated. FIG. 17B is a fluorescent image of the same field taken using the rhodamine filter with a 2 second exposure time. FIG. 17C is a brightfield image of lipid microbubbles with Nile red encapsulated. FIG. 17D is a fluorescent image of the same field of view using the rhodamine filter with a 2 second exposure time. FIG. 18A-D. Results of fluorescent protein conjugation experiment. Microbubbles were incubated with fluorescent NeutrAvidin protein. Scale bar is for all images. FIG. 18A Brightfield image of PSMs microbubbles with no biotinylated lipids. FIG. 18B Fluorescent image of same field as A FITC, 2 second exposure. FIG. 18C Brightfield image of microbubbles containing biotinylated lipids. FIG. 18D Fluorescent image of C FITC, 2 second exposure.

FIG. 19. Schematic of a microfluidic device for generating microbubbles. Lipids dispersed in solution were carried in a channel 50 μm in width, and a gas was carried in a channel 40 μm in width. Both the lipids and gas were focused through the orifice (6 μm) to create lipid-coated, gas-filled microbubbles. The structural formulas of the lipids used are given in the inset.

FIG. 20A. Stability of microbubbles containing 30% diacetylene. The images were taken 10 and 90 min after bubbles were produced. The images were taken 10 and 90 min after bubbles were produced. FIG. 20B. Stability of Vevo MicroMarker (VMM) microbubbles monitored using phase contrast microscopy and microbubble diameter histograms. FIG. 20C. Stability of nonpolymerizable shell microbubbles. The images were taken 10 and 90 min after bubbles were produced

FIG. 21. Ultrasound echogenecity (at 7.5 MHz) vs. time for a variety of microbubble shell materials: (+) 15% DA; (♦) 25% DA; (∘) 30% DA; (▪) VMM; (Δ) Nonpolymerizable lipids.

FIG. 22A. Targeted microbubbles on bovine smooth muscle cells. The microbubbles have the peptide sequence RGD (Arg-Gly-Asp) attached to them and bind to cells. FIG. 22B. Non-targeted microbubbles do not bind to bovine vascular smooth muscle cells.

### DETAILED DESCRIPTION OF THE INVENTION

The present invention relates to the fabrication and use of microbubbles. In some embodiments, the invention provides for the fabrication and use of polymerized shell lipid microbubbles (PSMs). In some embodiments the PSMs of the invention are used for ultrasound applications, offering the potential to tune the stability of a microbubble used as a diagnostic tool or drug/gene delivery vehicle subject to ultrasound. In some embodiments, the examples described herein demonstrate that by varying the amount of polymerized lipid in a monolayer, the mechanical strength of the monolayer can be increased. Without intending to be limited to any theory, this mechanical strength of a microbubble shell has various downstream effects for ultrasound contrast and/or delivery agents, most notably stability in an ultrasound field (resistance to destruction), ultrasound signal intensity, ability to squeeze through capillaries, and time-scale to break down in the body. By varying the mole fraction of polymerized lipid in a PSM, the system for particular applications can be fine-tuned and optimized.

Unless otherwise explained, all technical and scientific terms used herein have the same meaning as commonly understood by one of ordinary skill in the art to which this disclosure belongs. All references cited herein are all incorporated by reference herein in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention.

Other than in the operating examples, or where otherwise indicated, all numbers expressing quantities of ingredients or reaction conditions used herein should be understood as modified in all instances by the term “about” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below.

### Introduction

Recently, ultrasound contrast imaging has emerged as an advantageous imaging modality because of its low cost and wider availability compared to MRI. However, to date, targeted ultrasound contrast agents (USCA) have resulted in relatively low acoustic signal in vivo, and have thus found limited use in clinics. In some embodiments, the invention provides methods to improve signal by optimizing the targeted USCA through control of average size, polydispersity, and stability in the bloodstream, thereby increasing their potential applicability. Current techniques for producing microbubble contrast agents that involve sonication or agitation result in large polydispersity and batch-to-batch variation. Monodisperse microbubbles are desirable because they result in a more uniform acoustic response, greater echogenicity, and in the case of a contrast agent carrying a payload, a more selective drug release profile. In some embodiments, the invention provides for methods of producing monodisperse microbubbles. In some embodiments, microbubbles are produced by microfluidic flow focusing. Microfluidic flow focusing has the potential to create particles of narrow size distributions, which can be controlled by adjusting the flow rates of the two impinging fluids. In some embodiments, the invention provides a microfluidic flow focusing channel and methods for using the device for producing monodisperse microbubbles.

In some embodiments, by using monodisperse PSMs the resolution can be increased while tuning the shell rigidity to optimize microbubble properties for a given application. In addition to the proximal imaging applications, the technology can be used for drug delivery and gene delivery. By tuning the shell rigidity, and thus ultrasound stability, the PSMs might be engineered to be visualized at one ultrasound frequency and destroyed at a different frequency. Microbubble stability in ultrasound has been shown to directly affect gene transfer efficiency (Alter et al. 2009, Ultrasound Med. Biol. 35, 976-984).

In some embodiments, the microbubble shell composition improves stability in the bloodstream, thereby increasing blood circulation time. In some embodiments, the composition also affects ultrasound echogenicity by changing the surface elasticity, surface tension, or microstructure. Thus, without intending to be limited to any theory, the invention provides an enhanced acoustic response in the region of interest by using an optimal microbubble shell for targeted USCA.

In some embodiments, the invention provides a system using polymerizable lipids as the shell material, resulting in increased stability and enhanced ultrasonic contrast. In some embodiments, polymerizable lipids are lipids modified to contain a diacetylene group, which undergo rapid polymerization under UV exposure (e.g., 254 nm). In some embodiments, the microbubble shell comprises poly(ethylene glycol) (PEG) polymers tethered to the lipids. Without intending to be limited to any theory, the PEG polymers tethered to the lipids provide colloidal stability against aggregation and steric effects to block binding of opsonizing plasma proteins, which leads to increased lifetime in blood circulation. In some embodiments, the present invention describes methods to synthesize monodisperse polymerized lipid microbubbles, e.g., through microfluidic flow focusing, and to conjugate targeting molecules to the bubbles, e.g., using PEG-lipid tethered ligands. In some embodiments, the present invention provides microbubbles with improved and tunable acoustic properties and better controlled drug release relative to current techniques. In some embodiments, the methods, systems and compositions of the invention can be used, e.g., for molecular monitoring of plaque vulnerability and drug delivery to sites of inflammation.

Due to the shortcomings of current imaging technologies, much research has focused on the development of targeted imaging methods. However, ultrasound molecular imaging remains in the pre-clinical stages due to deficiencies in the systems developed. Various studies have demonstrated successful but limited capability of current targeted USCAs to detect increased surface ligand expression in vivo (Kaufmann et al. 2007, Circulation 116, 276-284; Weller et al. 2003, Circulation 108, 218-224). The USCAs used in these studies were fabricated using probe-tipped sonication, and authors expressed concern about low resolution. Recent studies focused on methods to increase resolution. Ferrante et al. conducted in vitro flow studies with dual targeted lipid microbubbles, resulting in increased binding capability (Ferrante et al. 2009, J. Control. Release 140, 100-107). Other methods to increase binding, such as the use of flexible spacer tether arms, are an active area of research (Klibanov 2005, Bioconjug. Chem. 16, 9-17). However, when using a polydisperse microbubble population only a small fraction of bound microbubbles is visualized, since relatively few of the microbubbles are of a size to produce significant ultrasound signal, while monodisperse populations offer much greater sensitivity (Talu et al. 2007, Mol. Imaging. 6, 384-392).

Thus, clinically available ultrasound contrast agents are limited to polydisperse, non-targeted microbubbles, which result in non-specific imaging, e.g., highlighting of the vasculature. In some embodiments, the invention provides targeted microbubbles. In some embodiments, the targeted microbubbles comprise polymerized lipids. In some embodiments, the targeted microbubbles comprise PEG polymers tethered to the lipids. In some embodiments, the microbubbles are prepared using a microfluidic flow focusing device. The targeted microbubbles described herein offer the potential to target specific molecular markers, (e.g., in the vasculature) revealing information about molecular makeup in addition to structure. Information about molecular makeup is crucial in many diagnostic applications, such as inflammation in atherosclerosis and angiogenesis in cancer. Additionally, by utilizing microbubbles as drug/gene delivery vehicles, a payload can be targeted to specific molecular markers and induced to release upon increased ultrasound stimulation, offering a means of image-guided drug delivery.

### Microbubbles

In one aspect, the present invention provides microbubbles. The term microbubbles refers to vesicles which are generally characterized by the presence of one or more membranes or walls or shells surrounding an internal void that is filled with a gas or precursor thereto. In some embodiments, the microbubbles comprise one or more lipids. The term lipids includes agents exhibiting amphipathic characteristics causing it to spontaneously adopt an organized structure in water wherein the hydrophobic portion of the molecule is sequestered away from the aqueous phase. In some embodiments, the microbubbles comprise polymerizable lipids. In some embodiments, the microbubbles comprise one or more lipids, at least one of which is polymerizable. In some embodiments the microbubbles comprise one or more gases inside a lipid shell. The microbubbles may optionally also contain targeting agents, therapeutic agents, and/or other functional molecules. The microbubbles of the invention may also include any other materials or combination thereof known to those skilled in the art as suitable for microbubble construction.

Lipids

In one aspect, the microbubbles of the invention comprise one or more lipid. The lipids used may be of natural and/or synthetic origin. Such lipids include, but are not limited to, fatty acids, lysolipids, dipalmitoylphosphatidylcholine, phosphatidylcholine, phosphatidic acid, sphingomyelin, cholesterol, cholesterol hemisuccinate, tocopherol hemisuccinate, phosphatidylethanolamine, phosphatidyl-inositol, lysolipids, sphingomyelin, glycosphingolipids, glucolipids, glycolipids, sulphatides, lipids with ether and ester-linked fatty acids, diacetyl phosphate, stearylamine, distearoylphosphatidylcholine, phosphatidylserine, sphingomyelin, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3.beta.-yloxy)-1-thio-.beta.-D-galactopyranoside, digalactosyldiglyceride, 6-(5-cholesten-3.beta.-yloxy)hexyl-6-amino-6-deoxy-1-thio-.beta.-D-galactop yranoside, 6-(5-cholesten-3.beta.-yloxy)hexyl-6-amino-6-deoxyl-1-thio-.alpha.-D-manno pyranoside, dibehenoyl-phosphatidylcholine, dimyristoylphosphatidylcholine, dilauroylphosphatidylcholine, and dioleoyl-phosphatidylcholine, and/or combinations thereof.

In some embodiments, the microbubbles of the invention comprise one or more polymerizable lipid. Examples of polymerizable lipids include but are not limited to, diyne PC and diynePE, for example 1,2-bis(10,12-tricosadiynoyl-sn-glycero-3-phosphocoline. In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of polymerizable lipids. In some embodiments, the microbubbles of the invention comprise at least 25% of polymerizable lipids. In some embodiments, the microbubbles of the invention comprise at least 50% of polymerizable lipids. In some embodiments, the polymerizable lipid may comprise a polymerizable group attached to a lipid molecule. The microbubbles may also contain lipids that are not polymerizable, lipids conjugated to a functional moiety (such as a targeting agent or a therapeutic agent), and lipids with a positive, negative, or neutral charge.

In some embodiments, the microbubbles of the invention comprise one or more neutral phospholipids. Examples of neutral phospholipids include, but are not limited to, hydrogenated phosphatidyl choline (HSPC), dipalmitoyl-, distearoyl- and diarachidoyl phosphatidylcholine (DPPC, DSPC, DAPC). In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of neutral phospholipids. In some embodiments, the microbubbles of the invention comprise at least 10% of neutral phospholipids. In some embodiments, the microbubbles of the invention comprise at least 30% of neutral phospholipids. In some embodiments, the microbubbles of the invention comprise at least 45% of neutral phospholipids.

In some embodiments, the microbubbles of the invention comprise one or more negatively charged phospholipids. Examples of negatively charged phospholipids include, but are not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG). In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged phospholipids. In some embodiments, the microbubbles of the invention comprise at least 2% of negatively charged phospholipids. In some embodiments, the microbubbles of the invention comprise at least 5% of negatively charged phospholipids. In some embodiments, the microbubbles of the invention comprise at least 10% of negatively charged phospholipids. In some embodiments, the microbubbles of the invention comprise at least 25% of negatively charged phospholipids. In some embodiments, the microbubbles of the invention comprise at least 30% of negatively charged phospholipids.

In some embodiments, the microbubbles of the invention comprise one or more reactive phospholipids. Examples of reactive phospholipids include, but are not limited to, phosphatidyl ethanolamine derivatives coupled to a polyethyleneglycol, a biotinyl, a glutaryl, a caproyl, a maleimide, a sulfhydral, a pyridinal disulfide or a succinyl amine. In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of reactive phospholipids. In some embodiments, the microbubbles of the invention comprise at least 2% of reactive phospholipids. In some embodiments, the microbubbles of the invention comprise at least 5% of reactive phospholipids. In some embodiments, the microbubbles of the invention comprise at least 10% of reactive phospholipids. In some embodiments, the microbubbles of the invention comprise at least 25% of reactive phospholipids. In some embodiments, the microbubbles of the invention comprise at least 30% of reactive phospholipids.

In some embodiments, the microbubbles of the invention comprise one or more lipids and phospholipids such as soy lecithin, partially refined lecithin, hydrogenated phospholipids, lysophosphate, phosphatidylcholine, phosphatidylethanolamine, phosphatidylserine, phosphatidylinositol, cardiolipin, sphingolipids, gangliosides, cerebrosides, ceramides, other esters analogue of phopshpatidylcholine (PAF, lysoPAF). In some embodiments, the microbubbles of the invention comprise one or more synthetic phospholipids such as L-a-lecithin (dilauroylphosphatidylcholine, dipalmitoylphosphatidylcholine, dilinoloylphosphatidylcholine, distearoylphosphatidylcholine, diarachidoylphosphatidylcholine); phosphatidylethanolamine derivatives, such as 1,2-diacyl-sn-glycero-3-phosphoethanolamine, 1-acyl-2-acyl-sn-glycero-3-phosphoethanolamine, dinitrophenyl- and dinitrophenylamino caproylphosphatidylethanolamine, 1,2-diacyl-sn-glycero-3-phosphoethanolamine-N-polyethylene glycol (PEG-PE), N-biotinyl-PE, N-caproylamine PE, N-dodecylamine-PE, N-MPB-PE, N-PDD-PE, N-succinyl-PE, N-glutaryl-PE; di-acetylenic lipids; phosphatidic acids (1,2-diacyl-sn-glycero-3-phosphate salt, 1-acyl-2-acyl-sn-glycero-3-phosphate sodium salt; phosphatidylserine such as 1,2-diacyl-snglycero-3-[phospho-L-serine] sodium salt, 1-acyl-2-acyl-sn-glycero-3-[phospho-L-serine] sodium salt, lysophosphatidic acid; cationic lipids such as 1,2-diacyl-3-trimethylammoniumpropane (TAP), 1,2-diacyl-3-dimethylammoniumpropane (DAP), N-[1-(2,3-dioleoyloxy) propyl-N,N′,N″-trimethylammonium chloride (DOTMA).

In some embodiments, the microbubbles of the invention comprise one or more lipids suitable for click chemistry, such as those containing azide and alkyne groups. In some embodiments, the microbubbles of the invention comprise one or more phospholipids with multivarious headgroups such as phosphatidylethanol, phosphatidylpropanol and phosphatidylbutanol, phosphatidylethanolamine-N-monomethyl, 1,2-disteraoyl(dibromo)-sn-glycero-3-phosphocoline. In some embodiments, the microbubbles of the invention comprise one or more phospholipids with partially or fully fluorinated cholesterol or cholesterol derivatives can be used in place of an uncharged lipid, as generally known to a person skilled in the art.

The surface of a microbubble may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as the therapeutic entity. Any species of lipid may be used, with the sole proviso that the lipid or combination of lipids and associated materials incorporated within the lipid matrix should form a monolayer phase under physiologically relevant conditions. As one skilled in the art will recognize, the composition of the microbubble may be altered to modulate the biodistribution and clearance properties of the resulting microbubbles.

Other useful lipids or combinations thereof apparent to those skilled in the art which are in keeping with the spirit of the present invention are also encompassed by the present invention. For example, carbohydrates bearing lipids may be employed for in vivo targeting as described in U.S. Pat. No. 4,310,505.

In some embodiments, the microbubbles of the invention comprise one or more polymerizable lipid. Polymerizable lipids that can be used in the present invention include those described in U.S. Pat. Nos. 5,512,294 and 6,132,764, and US publication No. 2010/0111840, incorporated by reference herein in their entirety.

In some embodiments, the hydrophobic tail groups of polymerizable lipids are derivatized with polymerizable groups, such as diacetylene groups, which irreversibly cross-link, or polymerize, when exposed to ultraviolet light or other radical, anionic or cationic, initiating species, while maintaining the distribution of functional groups at the surface of the microbubble. The resulting polymerized microbubble particle is stabilized against fusion with cell membranes or other microbubbles and stabilized towards enzymatic degradation. The size of the polymerized microbubbles can be controlled by the method described herein, but also by other methods known to those skilled in the art, for example, by extrusion.

Polymerized microbubbles may be comprised of polymerizable lipids, but may also comprise saturated and non-alkyne, unsaturated lipids. The polymerized microbubbles can be a mixture of lipids which provide different functional groups on the hydrophilic exposed surface. For example, some hydrophilic head groups can have functional surface groups, for example, biotin, amines, cyano, carboxylic acids, isothiocyanates, thiols, disulfides, .alpha.-halocarbonyl compounds, .alpha.,.beta.-unsaturated carbonyl compounds and alkyl hydrazines. These groups can be used for attachment of targeting agents, such as antibodies, ligands, proteins, peptides, carbohydrates, vitamins, nucleic acids or combinations thereof for specific targeting and attachment to desired cell surface molecules, and for attachment of therapeutic agents, such as drugs, nucleic acids encoding genes with therapeutic effect or radioactive isotopes. Other head groups may have an attached or encapsulated therapeutic agent, such as, for example, antibodies, hormones and drugs for interaction with a biological site at or near the specific biological molecule to which the polymerized microbubble particle attaches. Other hydrophilic head groups can have a functional surface group of diethylenetriamine pentaacetic acid, ethylenedinitrile tetraacetic acid, tetraazocyclododecane-1,4,7,10-tetraacetic acid (DOTA), porphoryin chelate and cyclohexane-1,2,-diamino-N,N′-diacetate, as well as derivatives of these compounds, for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

Large numbers of therapeutic agents may be attached to one polymerized microbubble that may also bear from several to about one thousand targeting agents for in vivo adherence to targeted surfaces. The improved binding conveyed by multiple targeting entities can also be utilized therapeutically to block cell adhesion to endothelial receptors in vivo. Blocking these receptors can be useful to control pathological processes, such as inflammation and metastatic cancer. For example, multi-valent sialyl Lewis X derivatized microbubbles can be used to block neutrophil binding, and antibodies against VCAM-1 on polymerized microbubbles can be used to block lymphocyte binding, e.g. T-cells.

The polymerized microbubble particle can also contain groups to control nonspecific adhesion and reticuloendothelial system uptake. For example, PEGylation of liposomes has been shown to prolong circulation lifetimes; see International Patent Application WO 90/04384.

The component lipids of polymerized microbubbles can be purified and characterized individually using standard, known techniques and then combined in controlled fashion to produce the final particle. The polymerized microbubbles can be constructed to mimic native cell membranes or present functionality, such as ethylene glycol derivatives, that can reduce their potential immunogenicity. Additionally, the polymerized microbubbles have a well-defined monolayer structure that can be characterized by known physical techniques such as transmission electron microscopy and atomic force microscopy.

In some embodiments, the microbubbles can be formed from lipid solutions. In some embodiments, the lipid solutions can be prepared using the following protocol. Lipids in powder form are dissolved in chloroform and combined at the desired mole fractions. Lipid mixtures can be a combination of commercially lipids as described herein. Upon combination, the mixtures are vortexed for several seconds to fully mix the lipids. The chloroform mixtures are then placed in a vacuum oven at 45° C. until the solvent evaporated. The lipids are then placed in vacuum for at least 2 additional hours at room temperature to completely remove the chloroform. The lipid powder is then dissolved in sterile filtered 10% glycerol, 10% propylene glycol, 80% DI water (10:10:80 aqueous solution). Lipid mixtures are dissolved in the 10:10:80 aqueous solution at a concentration of 5.32 pmol/mL. In some embodiments, the lipid powder can be dissolved in 5% glycerol, 5% propylene glycol, 90% DI water (5:5:90 aqueous solution). The lipid solutions are then heated to 60° C. and placed in a sonication bath for at least 30 minutes, until the solutions became clear. Lipid solutions containing different percentages (mole fraction) of PEG2000 conjugated lipids and varying amounts of polymerizable lipids can be used to produce the microbubbles.

Gases

In one aspect the invention provides gas filled microbubbles. In some embodiments the microbubbles comprise one or more gases inside a lipid shell. In some embodiments, the lipid shell comprises one or more polymerizable lipids. In some embodiments, the invention provides gas filled microbubbles substantially devoid of liquid in the interior. In some embodiments, the microbubbles are at least about 90% devoid of liquid, at least about 95% devoid of liquid, or about 100% devoid of liquid.

The microbubbles may contain any combination of gases suitable for the diagnostic or therapeutic method desired. For example, various biocompatible gases such as air, nitrogen, carbon dioxide, oxygen, argon, xenon, neon, helium, and/or combinations thereof may be employed. Other suitable gases will be apparent to those skilled in the art, the gas chosen being only limited by the proposed application of the microbubbles.

In some embodiments, the microbubbles contain gases with high molecular weight and size. In some embodiments, the microbubbles contain fluorinated gases, fluorocarbon gases, and perfluorocarbon gases. In some embodiments, the perfluorocarbon gases include perfluoropropane, perfluorobutane, perfluorocyclobutane, perfluoromethane, perfluoroethane and perfluoropentane, especially perfluoropropane. In some embodiments, the perfluorocarbon gases have less than six carbon atoms. Gases that may be incorporated into the microbubbles include but are not limited to: SF₆, CF₄, C₂F₆, C₃F₆, C₃F₈ C₄F₆, C₄F₈, C₄F₁₀, C₅F₁₀, C₅F₁₂, C₆F₁₂, (1-trifluoromethyl), propane (2-trifluoromethyl)-1,1,1,3,3,3 hexafluoro, and butane (2-trifluoromethyl)-1,1,1,3,3,3,4,4,4 nonafluor, air, oxygen, nitrogen, carbon dioxide, noble gases, vaporized therapeutic compounds, and mixtures thereof. The halogenated versions of hydrocarbons, where other halogens are used to replace F (e.g., Cl, Br, I) would also be useful.

In some embodiments, microbubbles containing gases with high molecular weight and size are used for ultrasound imaging purposes. Without intending to be limited to any theory, the gases with high molecular weight and size enhance ultrasound scattering.

In some embodiments, innocuous, low boiling liquids which will vaporize at body temperature or by the action of remotely applied energy pulses, like C₆F₁₄, are also usable as a volatile confinable microbubble component in the present invention. In some embodiments, the confined gases may be at atmospheric pressure or under pressures higher or lower than atmospheric; for instance, the confined gases may be at pressures equal to the hydrostatic pressure of the carrier liquid holding the gas filled microspheres.

In some embodiments, the microbubbles comprises mixtures of these gases, e.g., mixtures of perfluorocarbons with other perfluorocarbons and mixtures of perfluorocarbons with other gases, such as air, N₂, O₂, He. The first gas and the second gas can be respectively present in a molar ratio of about 1:100, 1:75, 1:50, 1:30, 1:20, 1:10, 1:5 or 1:1 to about 1000:1, 500:1, 250:1, 100:1, 75:1, 50:1, 10:1 or 5:1.

Targeting Agent

In some embodiments, the microbubbles of the invention comprise a targeting agent. The term targeting agent includes a molecule, macromolecule, or molecular assembly which binds specifically to a biological target. Any biologically compatible, natural or artificial molecule may be utilized as a targeting agent. Examples of targeting agents include, but are not limited to, amphetamines, barbiturates, sulfonamides, monoamine oxydase inhibitor substrates, antibodies (including antibody fragments and other antibody-derived molecules which retain specific binding, such as Fab, F(ab′)2, Fv, diabodies and scFv derived from antibodies); receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; ligands specific of cellular membranes; enzymes, lipids, nucleic acid ligands or aptamers, antihypertensive agents, neurotransmitters, aminoacids, oligopeptides, radio-sensitizers, steroids (e.g. estrogen and estradiol), mono- and carbohydrates (such as glucose derivatives), fatty acids, muscarine receptors and substrates (such as 3-quinuclidinyle benzilate), dopamine receptors and substrates (such as spiperone), one or more members of a specific binding interaction such as biotin or iminobiotin and avidin or streptavidin and peptides, and proteins capable of binding specific receptors.

In some embodiments, targeting agents are molecules which specifically bind to receptors or antigens found on vascular cells. In some embodiments, targeting agents are molecules which specifically bind to receptors, antigens or markers found on cells of angiogenic neovasculature or receptors, antigens or markers associated with tumor vasculature. The receptors, antigens or markers associated with tumor vasculature can be expressed on cells of vessels which penetrate or are located within the tumor, or which are confined to the inner or outer periphery of the tumor. In one embodiment, the invention takes advantage of pre-existing or induced leakage from the tumor vascular bed; in this embodiment, tumor cell antigens can also be directly targeted with agents that pass from the circulation into the tumor interstitial volume.

In some embodiments, the targeting agents target endothelial receptors, tissue or other targets accessible through a body fluid or receptors or other targets upregulated in a tissue or cell adjacent to or in a bodily fluid. Targeting agents attached to the polymerized microbubbles, or linking carriers of the invention include, but are not limited to, small molecule ligands, such as carbohydrates, and compounds such as those disclosed in U.S. Pat. No. 5,792,783 (small molecule ligands are defined herein as organic molecules with a molecular weight of about 1000 daltons or less, which serve as ligands for a vascular target or vascular cell marker); proteins, such as antibodies and growth factors; peptides, such as RGD-containing peptides (e.g. those described in U.S. Pat. No. 5,866,540), bombesin or gastrin-releasing peptide, peptides selected by phage-display techniques such as those described in U.S. Pat. No. 5,403,484, and peptides designed de novo to be complementary to tumor-expressed receptors; antigenic determinants; or other receptor targeting groups.

These head groups can be used to control the biodistribution, non-specific adhesion, and blood pool half-life of the polymerized microbubbles. For example, .beta.-D-lactose has been attached on the surface to target the asialoglycoprotein (ASG) found in liver cells which are in contact with the circulating blood pool. Glycolipids can be derivatized for use as targeting agents by converting the commercially available lipid (DAGPE) or the PEG-PDA amine into its isocyanate, followed by treatment with triethylene glycol diamine spacer to produce the amine terminated thiocarbamate lipid, which by treatment with the para-isothiocyanophenyl glycoside of the carbohydrate ligand produces the desired targeting glycolipids. This synthesis provides a water-soluble flexible spacer molecule spaced between the lipids that form the internal structure or core of the microbubble and the ligand that binds to cell surface receptors, allowing the ligand to be readily accessible to the protein receptors on the cell surfaces. The carbohydrate ligands can be derived from reducing sugars or glycosides, such as para-nitrophenyl glycosides, a wide range of which are commercially available or easily constructed using chemical or enzymatic methods.

In some embodiments, the targeting agent targets the microbubbles to a cell surface. Delivery of the therapeutic or imaging agent can occur through endocytosis of the microbubbles or through binding to the outside of the cell. Such deliveries are known in the art. See, for example, Mastrobattista, et al., Immunoliposomes for the Targeted Delivery of Antitumor Drugs, Adv. Drug Del. Rev. (1999) 40:103-27.

In some embodiments, the targeting agent is attached to a stabilizing entity. In one embodiment, the attachment is by covalent means. In another embodiment, the attachment is by non-covalent means. For example, antibody targeting agents may be attached by a biotin-avidin biotinylated antibody sandwich, to allow a variety of commercially available biotinylated antibodies to be used on the coated polymerized microbubble. Specific vasculature targeting agents of use in the invention include (but are not limited to) anti-VCAM-1 antibodies (VCAM=vascular cell adhesion molecule); anti-ICAM-1 antibodies (ICAM=intercellular adhesion molecule); anti-integrin antibodies (e.g., antibodies directed against αᵥβ₃ integrins such as LM609, described in International Patent Application WO 89/05155 and Cheresh et al. J. Biol. Chem. 262:17703-11 (1987), and Vitaxin, described in International Patent Application WO 9833919 and in Wu et al., Proc. Natl. Acad. Sci. USA 95(11):6037-42 (1998); and antibodies directed against P- and E-selectins, pleiotropin and endosialin, endoglin, VEGF receptors, PDGF receptors, EGF receptors, FGF receptors, MMPs, and prostate specific membrane antigen (PSMA). Additional targets are described by E. Ruoslahti in Nature Reviews: Cancer, 2, 83-90 (2002).

In one embodiment of the invention, the targeted agent is combined with an agent targeted directly towards tumor cells. This embodiment takes advantage of the fact that the neovasculature surrounding tumors is often highly permeable or “leaky,” allowing direct passage of materials from the bloodstream into the interstitial space surrounding the tumor. Alternatively, the targeted agent itself can induce permeability in the tumor vasculature. For example, when the agent carries a radioactive therapeutic agent, upon binding to the vascular tissue and irradiating that tissue, cell death of the vascular epithelium will follow and the integrity of the vasculature will be compromised.

In some embodiments, the targeting agents can be attached to the microbubbles using any feasible method known in the art such as carbodiimide, maleimide, disulfide, or biotin-streptavidin coupling.

Therapeutic Agent

In some embodiments, a therapeutic agent may be incorporated into the microbubbles. A variety of drugs and other bioactive compounds may be incorporated into the microbubbles, including antineoplastic agents, blood products, biological response modifiers, anti-fungals, hormones, vitamins, peptides, anti-tuberculars, enzymes, anti-allergic agents, anti-coagulators, circulatory drugs, metabolic potentiators, antivirals, antianginals, antibiotics, antiinflammatories, antiprotozoans, antirheumatics, narcotics, opiates, cardiac glycosides, neuromuscular blockers, sedatives, local anesthetics, general anesthetics, radioactive compounds, monoclonal antibodies, genetic material, and prodrugs.

In some embodiments, some of the bioactive compounds that may be incorporated into the microbubbles include genetic material such as nucleic acids, RNA, and DNA, of either natural or synthetic origin, including recombinant RNA and DNA and antisense RNA and DNA, genes carried on expression vectors such as plasmids, phagemids, cosmids, yeast artificial chromosomes (YACs), and defective or “helper” viruses, antigene nucleic acids, both single and double stranded RNA and DNA and analogs thereof; hormone products such as vasopressin, oxytocin, progestins, estrogens and antiestrogens and their derivatives, glucagon, and thyroid agents such as iodine products and anti-thyroid agents; biological response modifiers such as muramyldipeptide, muramyltripeptide, microbial cell wall components, lymphokines (e.g., bacterial endotoxins such as lipopolysaccharide, macrophage activation factor), subunits of bacteria (such as Mycobacteria, Corynebacteria), the synthetic dipeptide N-acetyl-muramyl-L-alanyl-Disoglutamine; cardiovascular products such as chelating agents and mercurial diuretics and cardiac glycosides; blood products such as parenteral iron, hemin, hematoporphyrins and their derivatives; respiratory products such as xanthine derivatives (theophylline & aminophylline); anti-infectives such as aminoglycosides, antifungals (amphotericin, ketoconazole, nystatin, griseofulvin, flucytosine (5-fc), miconazole, amphotericin B, ricin, and 13-lactam antibiotics (e.g., sulfazecin)), antibiotics such as penicillins, actinomycins and cephalosporins, antiviral agents such as Zidovudine, Ribavirin, Amantadine, Vidarabine, and Acyclovir, anti-helmintics, antimalarials, and antituberculous drugs; biologicals such as immune serums, antitoxins and antivenins, rabies prophylaxis products, bacterial vaccines, viral vaccines, toxoids; antineoplastics such as nitrosureas, hydroxyurea, procarbazine, Dacarbazine, mitotane, nitrogen mustards, antimetabolites (fluorouracil), platinum compounds (e.g., spiroplatin, cisplatin, and carboplatin), methotrexate, adriamycin, taxol, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, dactinomycin (actinomycin D), daunorubicin hydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), teniposide (VM-26), vinblastine sulfate (VLB), vincristine sulfate, bleomycin, bleomycin sulfate, methotrexate, adriamycin, arabinosyl, and alkylated derivatives of metallocene dihalides; mitotic inhibitors such as Etoposide and the Vinca alkaloids, radiopharmaceuticals such as radioactive iodine and phosphorus products; as well as interferons (Interferon α-2a and α-2b), Asparaginase and cyclosporins.

The bioactives may be incorporated into microbubbles singly or in combination with each other or with additional substances aimed to increase bioactive efficacy, such as adjuvants. Bioactives may be attached (covalently, such as by ester, substituted ester, anhydride, carbohydrate, polylactide, or substituted anhydride bonds, or non-covalently, such as by streptavidin linkages or ionic binding) to the surface of the microbubble directly to the lipids or to a moiety conjugated to the lipids, incorporated directly into the lipid membrane, or included in the interior of the microbubble, preferably in a vapor state.

In some embodiments, the microbubbles may include targeting agents to selectively concentrate the microbubbles to a particular region for imaging or therapeutic treatment. The targeted method is particularly suitable for diagnostic imaging to determine locations of tumors or atheroschlerotic plaques. Targeting also enhances local administration of toxic substances which, if not targeted, could (and would) otherwise cause significant secondary effects to other organs; such drugs include for instance Amphotericin B or NSAID's or drugs whose administration is required over prolonged periods such as Dexamethasone, insulin, vitamin E, etc. The method is also suitable for administration of thrombolytic agents such as urokinase or streptokinase, or antitumoral compounds such as Taxol etc.

Prodrugs and otherwise non-active agents may also be incorporated into microbubbles with an activator, such as a protease that removes an inactivating peptide, such that the agent and the activator are separated until the microbubble is dissolved. Alternatively, the agent and the activator may be incorporated into different populations of microbubbles and targeted to the same location so that the agent is selectively activated only at the target site.

Contrasting Agents

In some embodiments, the microbubbles described herein may also contain substances to enhance imaging, e.g., for diagnostics or to visualize treatment during drug delivery. Any suitable contrasting agent known in the art can be incorporated into the microbubbles. These can include paramagnetic gases, such as atmospheric air, which contains traces of oxygen 17, or paramagnetic ions such as Mn⁺², Gd⁺², and Fe⁺³, to be used as susceptibility contrast agents for magnetic resonance imaging. Microbubbles may contain radioopaque metal ions, such as iodine, barium, bromine, or tungsten, for use as x-ray contrast agents. Microbubbles may also be associated with other ultrasound contrast enhancing agents, such as SHU-454 or other microbubbles.

Linking Carriers

In some embodiments, the microbubbles comprise a linking carrier. The term linking carrier includes entities that serve to link agents, e.g., targeting agents and/or therapeutic agents, to the microbubbles. In some embodiments, the linking carrier serves to link a therapeutic agent and the targeting agent. In some embodiments, the linking carrier confers additional advantageous properties to the microbubbles. Examples of these additional advantages include, but are not limited to: 1) multivalency, which is defined as the ability to attach either i) multiple therapeutic agents and/or targeting agents to the microbubbles (e.g., several units of the same therapeutic agent, or one or more units of different therapeutic entities), which increases the effective “payload” of the therapeutic entity delivered to the targeted site; ii) multiple targeting agents to microbubble (e.g., one or more units of the same or different therapeutic agents); and 2) improved circulation lifetimes, which can include tuning the size of the particle to achieve a specific rate of clearance by the reticuloendothelial system.

In some embodiments, the linking carriers are biocompatible polymers (such as dextran) or macromolecular assemblies of biocompatible components (such as microbubbles). Examples of linking carriers include, but are not limited to, microbubbles, polymerized microbubbles, other lipid vesicles, dendrimers, polyethylene glycol assemblies, capped polylysines, poly(hydroxybutyric acid), dextrans, and coated polymers. A preferred linking carrier is a polymerized microbubble. Another preferred linking carrier is a dendrimer.

The linking carrier can be coupled to the targeting agent and/or the therapeutic agent by a variety of methods, depending on the specific chemistry involved. The coupling can be covalent or non-covalent. A variety of methods suitable for coupling of the targeting entity and the therapeutic entity to the linking carrier can be found in Hermanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

Stabilizing Entities

In some embodiments, the microbubbles contain a stabilizing entity. As used herein, “stabilizing” refers to the ability to impart additional advantages to the microbubbles, for example, physical stability, i.e., longer half-life, colloidal stability, and/or capacity for multivalency; that is, increased payload capacity due to numerous sites for attachment of targeting agents. Stabilizing entities include macromolecules or polymers, which may optionally contain chemical functionality for the association of the stabilizing entity to the surface of the microbubble, and/or for subsequent association of therapeutic agents and/or targeting agents. The polymer should be biocompatible with aqueous solutions. Polymers useful to stabilize the microbubbles of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolyner or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include proteins, such as albumin, polyalginates, and polylactide-glycolide copolymers, cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polyethylenes (such as, for example, polyethylene glycol, the class of compounds referred to as Pluronics®, commercially available from BASF, (Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of microbubbles which employ polymers to stabilize microbubble compositions will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

In some embodiments, the stabilizing entity is dextran. In some embodiments, the stabilizing entity is a modified dextran, such as amino dextran. In a further preferred embodiment, the stabilizing entity is poly(ethylene imine) (PEI). Without being bound by theory, it is believed that dextran may increase circulation times of microbubbles in a manner similar to PEG. Additionally, each polymer chain (i.e. aminodextran or succinylated aminodextran) contains numerous sites for attachment of targeting agents, providing the ability to increase the payload of the entire lipid construct. This ability to increase the payload differentiates the stabilizing agents of the present invention from PEG. For PEG there is only one site of attachment, thus the targeting agent loading capacity for PEG (with a single site for attachment per chain) is limited relative to a polymer system with multiple sites for attachment.

In some embodiments, the following polymers and their derivatives are used poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), polyvinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the microbubble or other molecule.

In some embodiments, the polymer may act as a hetero- or homobifunctional linking agent for the attachment of targeting agents, therapeutic entities, proteins or chelators such as DTPA and its derivatives.

In some embodiments, the stabilizing entity is associated with the microbubble by covalent means. In another embodiment, the stabilizing entity is associated with the microbubble by non-covalent means. Covalent means for attaching the targeting entity with the microbubbles are known in the art and described in the US publication 2010/0111840 entitled Stabilized Therapeutic and Imaging Agents, incorporated by reference herein in its entirety.

Noncovalent means for attaching the targeting entity with the microbubble include but are not limited to attachment via ionic, hydrogen-bonding interactions, including those mediated by water molecules or other solvents, hydrophobic interactions, or any combination of these.

In some embodiments, the stabilizing agent forms a coating on the microbubble.

In some embodiments, the microbubbles of the invention may also be linked to functional agents, such as poly(ethylene glycol) (PEG), that otherwise modify microbubble properties, such as aggregation tendencies, binding by opsonizing plasma proteins, uptake by cells, and stability in the bloodstream.

### Methods for Producing Microbubbles

The microbubbles described herein may be prepared in any suitable manner known to practitioners of the art, such as by sonication, vacuum drying, shaking of a lipid solution in the presence of a gas. In some embodiments, microbubbles described herein are prepared through microfluidic flow focusing of a gas into an aqueous solution of the encompassing lipids. If the microbubbles produced form a population heterogenous in size, the size of the microbubbles may be further adjusted, such as by extrusion through a filter with a fixed pore size. Upon assembly, microbubbles may be polymerized by UV light, e.g., for 2-5 minutes, or any other means for polymerization, depending on the crosslinking moiety on the polymerizable lipid(s). In some embodiments, microbubbles may be polymerized by UV light for 2, 5, 10, 15, 20, 30, 45, 50 or 60 minutes. In some embodiments, microbubbles may be polymerized by UV light for 1, 2, 5, 10, or 15 hours. The longer the UV exposure the more rigid the microbubble will be. The length of the UV exposure will vary depending on the composition and the application of the microbbubles. The UV wavelength can be in the range of UV wavelength: 200-400 nm.

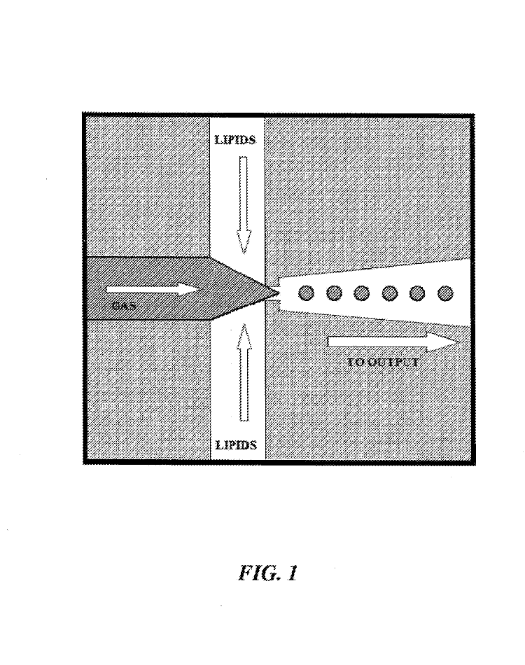

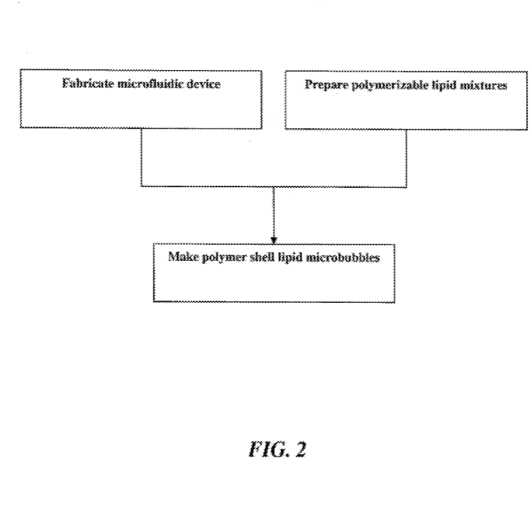

In some embodiments, the microbubbles described herein are produced by microfluidic flow focusing. Microfluidic flow focusing is a method for generating emulsions by flowing immiscible fluids through a small aperture, causing a pinching off of particles at regular intervals due to physical constraints. This method has been used successfully to generate microemulsions (Anna et al. 2003, Appl. Phys. Lett. 82, 364-366; Gafian-Calvo et al. 2001, Phys. Rev. Lett. 87, 274501; Garstecki et al. 2005, Phys. Rev. Lett. 94, 164501; Cubaud et al. 2005, Phys. Rev. E 72, 037302). It has been shown that viscosity controls size and distribution of particles (De Menech et al. 2008, J. Fluid Mech. 595, 141-161). Thus by varying the viscosity of the lipid solution, microbubble size and size distribution can be varied. For water in oil emulsions, flow rate and ratio of flows have been shown to control the size of particles (Anna et al. 2003, Appl. Phys. Lett. 82, 364-366). FIG. 1 shows the general schematic for a single emulsion through microfluidic flow focusing methods. In some embodiments, the gas phase is decafluorobutane, and the liquid phase is lipids in aqueous solution. The gas phase, decafluorobutane is forced through the aperture, creating gas-filled microbubbles. This method generates microbubbles with size distributions subject to control through various parameters.

In some embodiments, a microfluidic flow focusing is applied in order to generate polymerized shell microbubbles for use as ultrasound contrast agents. Current techniques use sonication to generate particles, resulting in large polydispersity due to lack of control. Using flow focusing, narrow distributions of particles can be generated, and size can be controlled through various parameters to generate particles of ideal size.

In an exemplary embodiment, a single emulsion microfluidic device was designed using AUTOCAD. The design was modified and scaled down from a previously described microfluidic flow-focusing device. The layout of the device orifice, where the emulsification occurs, is shown in FIG. 1. The gas enters the device through the 35 μm channel, and is focused through the orifice by the lipid solution, which flows through the 50 μm channels. This results in the pinching off of bubbles of gas, which are quickly stabilized by the formation of a monolayer of lipids at the gas-water interface, and exit through the 140 μm channel. The 75 μm channel could be used to form a second emulsion, or in this case was used to clear debris or clogs when they occurred.

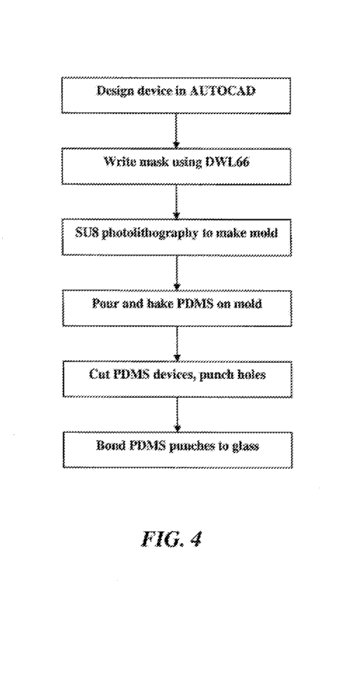

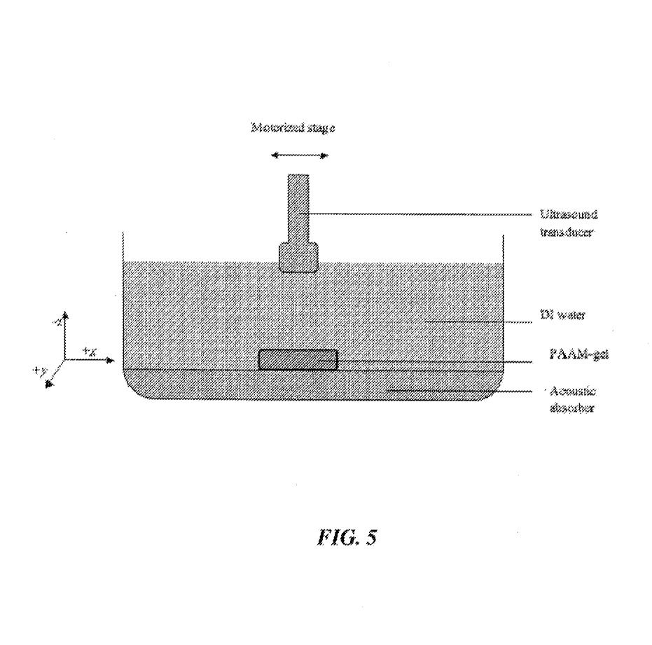

The process of advancing from an AUTOCAD design to an actual microfluidic device involves several steps. The basic steps that can be followed are shown in FIG. 4, attached. Following design of the device, a photomask of the design is fabricated in order to perform photolithography of the design. Next, using the photomask, photolithography techniques are used to pattern the design onto photoresist applied on a silicon wafer. This photoresist is then baked and surface treated to allow it to be used as a mold for the elastomeric polydimethylsiloxane (PDMS), the material used for the microfluidic devices. The PDMS is then poured and baked onto the micropatterned wafer. Finally, the PDMS is removed, cut into individual devices, hole-punched for portholes, and bonded to glass cover slips. Bonding between the glass coverslip and PDMS can be obtained by treating both surfaces in a plasma etcher and placing them together while applying light pressure. The result is a completed microfluidic device ready for use. These photolithography techniques are well described in the literature and, thus, known in the art.

In an exemplary embodiment, PDMS microfluidic devices are treated in a plasma asher for 5 minutes with high oxygen flow in order to make the surfaces hydrophilic to facilitate complete wetting of the interior of the devices. Polyethylene tubing (Becton Dickinson, Franklin Lakes, N.J.) is then inserted into the input portholes. Lipid solution is drawn into a 1 mL syringe, which is attached to the device in series with a 4 mm, 0.21 μm pore size syringe filter (Corning, Corning, N.Y.), a 23 gauge dispensing needle (McMaster-Carr, Elmhurst, Ill.), and tubing. Decafluorobutane gas (Synquest Laboratories, Alachua, Fla.) is attached to the device in a pressure-controlled manner. The canister is attached to a pressure regulator followed by a needle valve and a pressure meter. Finally, the gas is passed through a 0.2 μm syringe filter and into the device via tubing. Lipid solution is pumped into the device using a Harvard Apparatus syringe pump. Flow rate is controlled, and is varied in order to obtain microbubbles of different sizes, from approximately 1 μL/min up to μL/min Likewise, gas pressure is controlled using the pressure regulator, again varied to control the size and distribution of microbubbles produced, from 4PSI to 10 PSI. When the lipid solution is close to entering the device, the gas valve is opened to allow the gas to enter the device.

Microbubble production is monitored using an Axiovert 25 microscope (Zeiss, Oberkochen, Germany) and pictures are taken using a high-speed camera. The solution containing microbubbles is collected at the output port shown in FIG. 1.

### Polymerization of Bubbles

In some embodiments, the invention provides polymerized microbubbles. In some embodiments, the microbubbles comprise polymerizable lipids. In some embodiments, the microbubbles comprise one or more lipids, at least one of which is polymerizable. Polymerizable lipids may be polymerized by any suitable method known in the art. For example, polymerizable lipids may be polymerized addition of a catalyst to drive crosslinking, addition of a necessary linker molecule, or through photo-crosslinking, or with UV light. In some embodiments, polymerizable lipids may be polymerized with UV light.

In an exemplary embodiment, microbubbles in aqueous solution can be distributed in wells of a 96 well plate, and dispersed with a pipette prior to UV treatment. The plate can then be placed 6 inches directly under a germicidal 30W T8 UV lamp (General Electric, Fairfield, Conn.) and be subjected to 2-5 minutes, 30 minutes or even hours of UV light.

### General Methods

In one aspect, the present invention relates to the fabrication and use of microbubbles. One embodiment of the present invention involves the use of microbubbles for the classification, diagnosis, prognosis of a condition, determination of a condition stage, determination of response to treatment, monitoring and predicting outcome of a condition. Another embodiment of the invention involves the use of the microbubbles described herein in monitoring and predicting outcome of a condition. Another embodiment of the invention involves the use of the microbubbles described herein in drug screening, to determine which drugs may be useful in particular diseases. Another embodiment of the invention involves the use of the microbubbles described herein for the treatment of a condition.

The term “animal” or “animal subject” or “individual” as used herein includes humans as well as other mammals. In some embodiments, the methods involve the administration of one or more microbubbles for the treatment of one or more conditions. Combinations of agents can be used to treat one condition or multiple conditions or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying condition being treated. Also, a therapeutic benefit is achieved with the eradication or amelioration of one or more of the physiological symptoms associated with the underlying condition such that an improvement is observed in the patient, notwithstanding that the patient may still be afflicted with the underlying condition. For prophylactic benefit, the compositions may be administered to a patient at risk of developing a particular disease, or to a patient reporting one or more of the physiological symptoms of a disease, even though a diagnosis of this disease may not have been made.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

In some embodiments, the invention provides targeted ultrasound molecular imaging and drug delivery. In some embodiments, the invention provides the use of the microbubbles described herein for non-specific ultrasound diagnostics or for gene delivery applications.

In some embodiments, the invention provides methods for producing monodisperse size distribution population of microbubbles. In some embodiments, a monodisperse size distribution population of microbubbles results in greater ultrasound contrast (higher resolution), uniform ultrasound response, and more specific frequency excitation relative to polydisperse populations. In some embodiments, polymerized microbubbles produced using traditional means such as sonication, which result in a polydisperse size distribution, would offer the same novel benefits of the polymerized microbubble shell. Many methods of producing lipid shelled microbubbles have been described in the literature, which result in various size distributions, but none have been used to synthesize polymerized shell lipid microbubbles. Thus, one embodiment of this invention lies in the use of polymerized lipids to make a microbubble and their use as ultrasound contrast/delivery vehicles.

In some embodiments, the invention provides for the fabrication and use of polymerized shell lipid microbubbles (PSMs). PSMs of this invention may be used for a variety of diagnostic and therapeutic purposes, both in vivo and in vitro. In some embodiments the PSMs of the invention are used for ultrasound applications, offering the potential to tune the stability of a microbubble used as a diagnostic tool or drug/gene delivery vehicle subject to ultrasound. In some embodiments, the examples described herein demonstrate that by varying the amount of polymerized lipid in a monolayer, the mechanical strength of the monolayer can be increased. The PSMs may be untargeted or optionally contain targeting agents that specifically recognize target site(s), allowing for selectively enhancing imaging or therapeutic delivery of one or more therapeutic agents.

In some embodiments, the PSMs may be used to enhance imaging for diagnostic purposes. The microbubbles of this invention may also contain substances to enhance imaging, e.g. for diagnostics or to visualize treatment during drug delivery. These can include paramagnetic gases, such as atmospheric air, which contains traces of oxygen 17, or paramagnetic ions such as Mn⁺², Gd⁺², and Fe⁺³, to be used as susceptibility contrast agents for magnetic resonance imaging. Microbubbles may contain radioopaque metal ions, such as iodine, barium, bromine, or tungsten, for use as x-ray contrast agents. Microbubbles may also be associated with other ultrasound contrast enhancing agents, such as SHU-454.

Yet another aspect of the invention involves using the reflective characteristics of PSMs as a shield to prevent a HIFU beam from reaching underlying non-target tissue during ultrasound-based therapeutic heat treatment.

The use of polymerizable lipids to produce gas-filled microbubbles and their use, e.g., ultrasound diagnostic and therapeutic applications, provides several advantages. The use of polymerizable lipids in the making of the microbubbles for use with clinical ultrasound offer control over properties of a contrast agent or drug/gene delivery vehicle, allowing one to modulate and optimize properties for a given application. Examples of some microbubbles properties are echogenicity, mechanical elasticity, reduced microbubble aggregation and non-reactiveness with respect to the immune system uptake, increasing the amount of circulation time, and attaching targeting ligand.

The advantages of the embodiments of the methods and PSM described herein include the ability to adapt the shell properties for a given application and the ability to modulating properties to allow for ultrasound imaging at one frequency and drug release at another. Current technologies suffer from well-known deficits and the field of ultrasound targeted imaging is ripe for improvement and could receive widespread use due to the high availability and absence of radiation of ultrasound relative to other imaging modalities.

In some embodiments, the invention provides methods that can be used to produce polymerized shell lipid microbubbles of varying size and distribution. In some embodiments, the invention provides the use of polymerized lipids for ultrasound applications in diagnostics and therapeutics. In some embodiments, the invention provides for varying lipid formulations, adding PEG to the lipid head groups, or modifications of the polymerized lipid group to obtain different properties. In some embodiments, the microbubbles can be conjugated with antibodies or peptides for targeting applications through various chemistries, or used for non-specific purposes without a targeting moiety. In some embodiments, various methods could be used for the loading of the payload, such as using a lipophilic drug that localizes in the lipid shell or covalently linking a drug to the shell for delivery applications in drug and gene therapies.

In some embodiments, provided herein described is a method for the formation of gas-filled microbubbles with a polymerized lipid shell and their use in ultrasound diagnostics and therapeutics, where it is shown the capability to control properties of special interest for these technologies. In some embodiments, the microbubbles contain fluorinated gases, fluorocarbon gases, and perfluorocarbon gases. Examples of perfluorocarbon gases include, but are not limited to, perfluoropropane, perfluorobutane, perfluorocyclobutane, perfluoromethane, perfluoroethane and perfluoropentane, perfluoropropane, and/or a combination thereof.

In some embodiments, uniformly sized gas bubbles with a polymerized lipid shell are synthesized using a flow-focusing microfluidic device. In other embodiments, it is also possible to synthesize the polymerized lipid shell microbubbles through other techniques, and these microbubbles would have similar characteristics as those produced by a flow-focusing microfluidic device.

In some embodiments, the invention provides polymerized lipid microbubbles with increased stability. In some embodiments, the increased stability is achieved by increasing the mole fraction of polymerized lipids. In some embodiments, the microbubbles of the invention remain intact after 2, 3, 4, 5, 7, 8, 9 10, 20, 30, 40, 50, 60, 70, 90 minutes. In some embodiments, the microbubbles of the invention remain intact after 2, 3, 4, 5, 7, 8, 9 10, 15, 20, 30, 40, 50, 60, 70, 90 hours. In some embodiments, the microbubbles of the invention remain intact after two days.

In some embodiments, the microbubbles of the invention are used as ultrasound contrast agents. In some embodiments, the microbubbles retain at least 30%, 35%, 40%, 45%, 50%, 60%, 70%, 75%, 80%, or 90% of its signal after two seconds of ultrasound insonation. In some embodiments, the microbubbles retain at least 90% of its signal after two seconds of ultrasound insonation. In some embodiments, the microbubbles retain at least 30%, 35%, 40%, 45%, 50%, 60%, 70%, 75%, 80%, or 90% of its signal after 1, 2, 3, 4, 5, 6, 7, 8, 9, 10, 15 minutes of ultrasound insonation. In some embodiments, the microbubbles retain at least 75% of its signal after 10 minutes of ultrasound insonation. In some embodiments, the microbubbles retain at least 75% of its signal after 15 minutes of ultrasound insonation. In some embodiments, the microbubbles retain at least 90% of its signal after 10 minutes of ultrasound insonation. In some embodiments, the microbubbles retain at least 90% of its signal after 15 minutes of ultrasound insonation. In some embodiments, the microbubbles retain at least 75% of its signal after 1 hr or more of ultrasound insonation. In some embodiments, the microbubbles retain at least 75% of its signal after 2 hr or more of ultrasound insonation. In some embodiments, the microbubbles retain at least 90% of its signal after 1 hr or more of ultrasound insonation. In some embodiments, the microbubbles retain at least 90% of its signal after 2 hr or more of ultrasound insonation. In some embodiments, the microbubbles retain at least 90% of its signal after 3 or 4 hr. In some embodiments, the microbubbles are selectively destroyed at frequency other than the imaging frequency.

In some embodiments, the microbubbles of the invention have increased circulation time. In some embodiments, the microbubbles of the invention remain intact in circulation after 2, 3, 4, 5, 7, 8, 9 10, 20, 30, 40, 50, 60, 70, 90 minutes. In some embodiments, the microbubbles of the invention remain intact in circulation after 2, 3, 4, 5, 7, 8, 9 10, 15, 20, 30, 40, 50, 60, 70, 90 hours. In some embodiments, the microbubbles of the invention remain intact in circulation after two days. In some embodiments, the microbubbles of the invention are cleared from the system after the targeted microbubbles have been destroyed, e.g., by ultrasound insonication after 3 or 4 hours of administration.

In some embodiments, the microbubbles of the invention have increased half life. In some embodiments, the microbubbles of the invention have a half life of 2, 3, 4, 5, 7, 8, 9 10, 20, 30, 40, 50, 60, 70, 90 minutes. In some embodiments, the microbubbles of the invention have a half life of 2, 3, 4, 5, 7, 8, 9 10, 15, 20, 30, 40, 50, 60, 70, 90 hours. In some embodiments, the microbubbles of the invention have a half life of two days. In some embodiments, the microbubbles of the invention have a half life of two hours. In some embodiments, 90% of the microbubbles remain intact after 1 hr but are completely cleared from the system after 3 to 4 hr after administration. In some embodiments, the microbubbles are completely cleared from the system not before 1 hr but are cleared after 3 to 4 hr after administration.

In some embodiments, the invention provides microbubbles comprising a therapeutic agent. In some embodiments, the ratio by weight of therapeutic agent to lipid can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1. In some embodiments, the ratio by weight of therapeutic agent to lipid is 1:2. In some embodiments, the ratio by weight of therapeutic agent to lipid is 1:1. In some embodiments, the therapeutic agent is in an oil:drug phase on the outer edge of the gas layer because both the oil and the therapeutic agent are hydrophobic. In some embodiments the ratio of drug to oil is 1:2. In some embodiments the ratio of drug to oil is 1:1.

In some embodiments, the microbubbles of the invention retain the therapeutic agent under physiological conditions. In some embodiments, the microbubbles of the invention retain 50%, 55%, 60%, 70%, 80%, 90%, 95%, 99% of the therapeutic agent. In some embodiments, the microbubbles of the invention retain at least 70% of the therapeutic agent. In some embodiments, the microbubbles of the invention retain 80% of the therapeutic agent. In some embodiments, the microbubbles of the invention retain 90% of the therapeutic agent. In some embodiments, the microbubbles of the invention retain 100% of the therapeutic agent.

In some embodiments, without intending to be limited to any theory, polymerization prevents the therapeutic agent leakage for days under physiological conditions. The partially or completely polymerized microbubbles of the invention are stable against leakage yet capable of instantaneous release for remote controlled drug delivery. Polymerization increases not only the stability in solution but also the stability under ultrasound (dissolution rate), offering greater mechanical stability to help counter microbubble destruction. The dissolution rate is tunable by controlling the amount of polymer in the shell.

In some embodiments, the invention provides microbubbles to be imaged at one ultrasound frequency and release their payload at another. In some embodiments, microbubble shell properties are optimized to maximize efficiency for a given application, increasing or decreasing stiffness to maximize binding at a target site or modulating stability to optimize gene delivery.

In some embodiments, the present invention provides for a microbubble comprising a polymerized lipid shell and a gas, wherein the gas is encased with the shell. In some embodiments, the microbubbles of the invention comprise one or more polymerizable lipid. Examples of polymerizable lipids include but are not limited to, diyne PC and diynePE, for example 1,2-bis(10,12-tricosadiynoyl-sn-glycero-3-phosphocoline. In one embodiment, the polymerized lipid shell of the microbubble comprises at least one polymerizable lipid and at least one non-polymerizable lipid and has a percentage of about 5-50% polymerizable lipid. In some embodiments, the percentage of polymerizable lipid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of the total lipid mixture of making the microbubbles. In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of polymerizable lipids. In some embodiments, the microbubbles of the invention comprise at least 25% of polymerizable lipids. In some embodiments, the microbubbles of the invention comprise at least 50% of polymerizable lipids. In one embodiment, the at least one polymerizable lipid is a diacetylenic lipid

In some embodiments, the microbubbles of the invention comprise one or more negatively charged phospholipids. Examples of negatively charged phospholipids include, but are not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG).

In one embodiment, the at least one non-polymerizable lipid is selected group the group of L-α-phosphatidylcholine, PE-PEG2000 (1,2-distearoyl-sn-glycero-3-phosphoethanolamine-N-[methoxy(polyethylene glycol)-2000 or PE-PEG2000-biotin. In one embodiment, the polymerized lipid shell comprises a percentage of PEGylated lipid between about 1-20%. In some embodiments, the percentage of PEGylated lipid in the microbubble is 1, 2, 3, 4, 5, 6, 7, 8, 9, 10, 11, 12, 13, 14, 15, 16, 17, 18, 19, or 20. In one embodiment, the lipid is non-polymerizable and PEGylated. In one embodiment, the lipid is polymerizable and PEGylated.

In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged lipids. In some embodiments, the microbubbles of the invention comprise at least 2% of negatively charged lipids. In some embodiments, the microbubbles of the invention comprise at least 5% of negatively charged lipids. In some embodiments, the microbubbles of the invention comprise at least 10% of negatively charged lipids. In some embodiments, the microbubbles of the invention comprise at least 25% of negatively charged lipids. In some embodiments, the microbubbles of the invention comprise at least 30% of negatively charged lipids.

In some embodiments, the microbubbles of the invention comprise at least 2% of negatively charged lipids and at least 5-50% of a polymerizable lipid. In some embodiments, the microbubbles of the invention comprise at least 5% of negatively charged lipids and at least 5-50% of a polymerizable lipid. In some embodiments, the microbubbles of the invention comprise at least 10% of negatively charged lipids and at least 5-50% of a polymerizable lipid. In some embodiments, the microbubbles of the invention comprise at least 25% of negatively charged lipids and at least 5-50% of a polymerizable lipid. In some embodiments, the microbubbles of the invention comprise at least 30% of negatively charged lipids and at least 5-50% of a polymerizable lipid. In some embodiments, the microbubbles of the invention comprise the same percentage of negatively charged lipids and polymerizable lipids. In some embodiments, the negatively charged lipid and the polymerizable lipid is the same. In some embodiments, the microbubbles of the invention comprise at least two negatively charged lipids, but only one of the two negatively charged lipids is polymerizable.

In one embodiment, the gas of the microbubble is a heavy gas.

In one embodiment, the heavy gas is a perfluorocarbon. In another embodiment, the gas of the microbubble is a mixture of at least two perfluorocarbons. Perfluorocarbons (PFCs) are fluorocarbons, compounds derived from hydrocarbons by replacement of hydrogen atoms by fluorine atoms. PFCs are made up of carbon and fluorine atoms only, such as octafluoropropane, perfluorohexane and perfluorodecalin. A perfluorocarbon can be arranged in a linear, cyclic, or polycyclic shape. Perfluorocarbon derivatives are perfluorocarbons with some functional group attached, for example perfluorooctanesulfonic acid. Perfluorocarbon derivatives can be very different from perfluorocarbons in their properties, applications and toxicity. Other examples of perfluorocarbons are tetrafluoromethane, hexafluoroethane, octafluoropropane (perfluoropropane), perfluorocyclobutane, perfluoro-n-butane, and perfluoro-iso-butane. In some embodiments, the perfluorocarbon is decafluorobutane.

In some embodiments, the microbubble has a diameter size range that is about 3 ng-5 μm. In some embodiments, the microbubble has a diameter size range that is about 50 ng-5 μm. In some embodiments, the microbubble has a diameter size range that is about 1 μg-5 μm. In some embodiments, the microbubble has a diameter size range that is about 3-5 μm. In some embodiments, the microbubble has a diameter size range of about 2 μm. In some embodiments, the microbubble has a diameter size of about 3 μm. In another embodiment, the microbubble has a diameter size of about 4 μm. In one embodiment, the microbubble has a diameter size of about 3-5 μm. In other embodiments, the microbubble has a diameter size of about 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 μm.

In one embodiment, the microbubble is conjugated with a ligand and the conjugation is by any way of the tethering the ligand to the lipid shell. Methods of tethering ligands to microbubbles are well known in the art, e.g. using carbodiimide, maleimide, or biotin-streptavidin coupling (Klibanov 2005, Bioconjug. Chem. 16, 9-17). Biotin-streptavidin is the most popular coupling strategy because biotin's affinity for streptavidin is very strong and it is easy to label ligands with biotin. In some embodiments, ligands include monoclonal antibodies and other ligands that bind to receptors (e.g. VCAM-1, ICAM-1, E-selection) expressed by the cell type of interest, inflammatory cells.

In one embodiment, the microbubbles encapsulate a therapeutic agent (e.g., drug, a chemical) or any entity within the shell. In some embodiments, the therapeutic agent or entity within the shell is delivered to a target location by way of the microbubble.

In one embodiment, the microbubble is UV treated for about 2-5 minutes after fabrication to polymerize the lipid shell. It is understood that one can UV treat the formed microbubbles for a time period of anywhere from 2.0 min to several hours in order to achieve various/desired level of polymerization in the shell. In some embodiments, the microbubble is UV treated for about 2, 5, 10, 15, 20, 30, 45, 50 or 60 minutes. In some embodiments, the microbubble is UV treated for about 1, 2, 5, 10, or 15 hours. The UV wavelength can be in the range of UV wavelength: 200-400 nm. The shell material affects microbubble mechanical elasticity. The more elastic the material, the more acoustic energy it can withstand before bursting (McCulloch et al., 2000, J Am Soc Echocardiogr. 13: 959-67). The level of polymerization of the shell affects the mechanical elasticity. By varying the UV treatment timing, the amount of polymerization of the shell can be adjusted, e.g. 2 or 3 min for lower polymerization, 4-30 min for higher polymerization. In one embodiment, the microbubble is UV treated for about 2 minutes. In another embodiment, the microbubble is UV treated for about 3 minutes. In another embodiment, the microbubble is UV treated for about 4 minutes. In another embodiment, the microbubble is UV treated for about 5 minutes. In other embodiments, the microbubble is UV treated for about 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 minutes. In another embodiment, the microbubble is UV treated for about 10 minutes. In another embodiment, the microbubble is UV treated for about 20 minutes. In another embodiment, the microbubble is UV treated for about 30 minutes. In another embodiment, the microbubble is UV treated for about 60 minutes. In another embodiment, the microbubble is UV treated for about 2 hours.

In one embodiment, the microbubble has an absorbance at a wavelength between about 400-580 μm. In some embodiments, the microbubble has an absorbance at a wavelength of about 400, 410, 420, 430, 440, 450, 460, 470, 480, 490, 500, 510, 520, 530, 540, 550, 560, 570, or 580 μm. In one embodiment, the absorbance at a wavelength between about 400-580 nm is an indication of the successful polymerization of the polymerizable lipid forming the shell of the microbubble. This is especially so when the polymerizable lipid is a diacetylenic lipid. In another embodiment, the microbubble appears to be blue or purple, wherein blue indicates one form of polymerized diacetylenic lipid and purple indicates a mixture of a red and a blue form of polymerized diacetylenic lipid.

In another embodiment, the present invention provides for a collection of microbubbles comprising gas-filled polymerized shell lipid microbubbles, wherein the microbubbles in the collection are monodispersed and are within a micrometer size range. In one embodiment, the microbubbles of the collection have all the characteristics of a microbubble described herein. In some embodiments, the collection of monodispersed microbubbles is generated by a microfluidic flow focusing method.

In one embodiment, the collection of microbubbles is monodispersed, and the monodisperity is about 20% of an average size of the microbubbles in the collection. In one embodiment, the collection of microbubbles is monodispersed, and the monodisperity is about 15% of an average size of the microbubbles in the collection. In one embodiment, the collection of microbubbles is monodispersed, and the monodisperity is about 10% of an average size of the microbubbles in the collection. In one embodiment, the collection of microbubbles is monodispersed, and the monodisperity is about 5% of an average size of the microbubbles in the collection. In one embodiment, the collection of microbubbles is monodispersed, and the monodisperity is about 1% of an average size of the microbubbles in the collection.

In some embodiments, 90% of the microbubbles in the collection remain intact after 2, 3, 4, 5, 7, 8, 9 10, 20, 30, 40, 50, 60, 70, 90 minutes. In some embodiments, 90% of the microbubbles in the collection remain intact after 2, 3, 4, 5, 7, 8, 9 10, 15, 20, 30, 40, 50, 60, 70, 90 hours. In some embodiments, 90% of the microbubbles in the collection remain intact after two days. In some embodiments, 80% of the microbubbles in the collection remain intact after 2, 3, 4, 5, 7, 8, 9 10, 20, 30, 40, 50, 60, 70, 90 minutes. In some embodiments, 80% of the microbubbles in the collection remain intact after 2, 3, 4, 5, 7, 8, 9 10, 15, 20, 30, 40, 50, 60, 70, 90 hours. In some embodiments, 80% of the microbubbles in the collection remain intact after two days. In some embodiments, 50% of the microbubbles in the collection remain intact after 2, 3, 4, 5, 7, 8, 9 10, 20, 30, 40, 50, 60, 70, 90 minutes. In some embodiments, 50% of the microbubbles in the collection remain intact after 2, 3, 4, 5, 7, 8, 9 10, 15, 20, 30, 40, 50, 60, 70, 90 hours. In some embodiments, 50% of the microbubbles in the collection remain intact after two days. In some embodiments, 90% of the microbubbles in the collection remain intact after 90 minutes. In some embodiments, 50% of the microbubbles in the collection remain intact after 15 hours. In some embodiments, 50% of the microbubbles in the collection remain intact after two days.

In some embodiments, the collection of microbubbles of the invention is used as ultrasound contrast agents. In some embodiments, the collection of microbubbles retains at least 30%, 35%, 40%, 45%, 50%, 60%, 70%, 75%, 80%, or 90% of its signal after two seconds of ultrasound insonation. In some embodiments, the collection of microbubbles retains at least 90% of its signal after two seconds of ultrasound insonation. In some embodiments, the collection of microbubbles retains at least 30%, 35%, 40%, 45%, 50%, 60%, 70%, 75%, 80%, or 90% of its signal after 1, 2, 3, 4, 5, 6, 7, 8, 9, 10, 15 minutes of ultrasound insonation. In some embodiments, the collection of microbubbles retains at least 75% of its signal after 10 minutes of ultrasound insonation. In some embodiments, the collection of microbubbles retains at least 75% of its signal after 15 minutes of ultrasound insonation. In some embodiments, the collection of microbubbles retains at least 90% of its signal after 10 minutes of ultrasound insonation. In some embodiments, the collection of microbubbles retains at least 90% of its signal after 15 minutes of ultrasound insonation. In some embodiments, the collection of microbubbles retains at least 75% of its signal after 1 hr or more of ultrasound insonation. In some embodiments, the collection of microbubbles retains at least 75% of its signal after 2 hr or more of ultrasound insonation. In some embodiments, the collection of microbubbles retains at least 90% of its signal after 1 hr or more of ultrasound insonation. In some embodiments, the collection of microbubbles retains at least 90% of its signal after 2 hr or more of ultrasound insonation. In some embodiments, the collection of microbubbles retains at least 90% of its signal for at least 30 minutes for the diagnostic imaging session and then the desired targeted microbubbles are destroyed. The rest of the microbubbles are cleared from the system about 3 to 4 hours later without them being destroyed.

In some embodiments, the collection of microbubbles of the invention has increased circulation time. In some embodiments, the collection of microbubbles of the invention remains intact in circulation after 2, 3, 4, 5, 7, 8, 9 10, 20, 30, 40, 50, 60, 70, 90 minutes. In some embodiments, the collection of microbubbles of the invention remains intact in circulation after 2, 3, 4, 5, 7, 8, 9 10, 15, 20, 30, 40, 50, 60, 70, 90 hours. In some embodiments, the collection of microbubbles of the invention remains intact in circulation after two days. In some embodiments, 90% of the collection of microbubbles of the invention remains intact in circulation after about 3 to 4 hours.

In some embodiments, the collection of microbubbles of the invention has increased half life. In some embodiments, the collection of microbubbles of the invention has a half life of 2, 3, 4, 5, 7, 8, 9 10, 20, 30, 40, 50, 60, 70, 90 minutes. In some embodiments, the collection of microbubbles of the invention has a half life of 2, 3, 4, 5, 7, 8, 9 10, 15, 20, 30, 40, 50, 60, 70, 90 hours. In some embodiments, the collection of microbubbles of the invention has a half life of two days. In some embodiments, of the collection of microbubbles of the invention remains intact in circulation after about 3 to 4 hours, but then are cleared from the system.

In some embodiments, the average size of the microbubbles in the collection is between about 3 nm-5 μm. In some embodiments, the average size of the microbubbles in the collection is between about 50 nm-5 μm. In some embodiments, the average size of the microbubbles in the collection is between about 1 μm-5 μm. In some embodiments, the average size of the microbubbles in the collection is between about 3-5 μm. In some embodiments, the average size of the microbubbles in the collection is about 2 μm. In some embodiments, the average size of the microbubbles in the collection is about 3 min.

In another embodiment, the average size of the microbubbles in the collection is about 4 μm. In one embodiment, the average size of the microbubbles in the collection is between about 3-5 μm. In other embodiments, the average size of the microbubbles in the collection is about 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 μm.

In some embodiments, the microbubbles of the collection comprise at least 25% of polymerizable lipids. In some embodiments, the microbubbles of the collection comprise at least 50% of polymerizable lipids. In one embodiment, the at least one polymerizable lipid is a diacetylenic lipid

In some embodiments, the microbubbles of the collection comprise one or more negatively charged lipids. In one embodiment, the microbubbles of the collection comprise a percentage of PEGylated lipid between about 1-20%.

In some embodiments, the microbubbles of the collection comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged lipids. In some embodiments, the microbubbles of the collection comprise at least 2% of negatively charged lipids. In some embodiments, the microbubbles of the collection comprise at least 5% of negatively charged lipids. In some embodiments, the microbubbles of the collection comprise at least 10% of negatively charged lipids. In some embodiments, the microbubbles of the collection comprise at least 25% of negatively charged lipids. In some embodiments, the microbubbles of the collection comprise at least 30% of negatively charged lipids.

In some embodiments, the microbubbles of the collection comprise at least 2% of negatively charged lipids and at least 5-50% of a polymerizable lipid. In some embodiments, the microbubbles of the collection comprise at least 5% of negatively charged lipids and at least 5-50% of a polymerizable lipid. In some embodiments, the microbubbles of the collection comprise at least 10% of negatively charged lipids and at least 5-50% of a polymerizable lipid. In some embodiments, the microbubbles of the collection comprise at least 25% of negatively charged lipids and at least 5-50% of a polymerizable lipid. In some embodiments, the microbubbles of the collection comprise at least 30% of negatively charged lipids and at least 5-50% of a polymerizable lipid. In some embodiments, the microbubbles of the collection comprise the same percentage of negatively charged lipids and polymerizable lipids. In some embodiments, the negatively charged lipid and the polymerizable lipid is the same.

In some embodiments, the present invention provides for a method of making a microbubble comprising: (a) microfluidic flow focusing a mixture of polymerizable lipid and standard non-polymerizable lipid and a gas through an aperture to form micrometer microbubbles and (b) UV treating the microbubbles of step a to polymerize the polymerizable lipid.

### Drug Delivery

In some embodiments, one or more therapeutic agents may be attached to the surface of the microbubble, incorporated in the lipid layer, or trapped within the lipid shell. Microbubbles may further include a targeting agent or agents that recruit the microbubble to a target site. In some embodiments, to rupture the microbubbles and release the therapeutic agent(s), the microbubbles may be irradiated with an energy beam, preferably ultrasonic. The frequency of the ultrasonic irradiation required to break the microspheres may vary from about 0.3 to 3 MHz. When ultrasound is applied at a frequency corresponding to the peak resonant frequency of the microbubbles, the microbubbles will rupture and release their contents. Rupture efficiency can also be enhanced by increasing the duration or the strength of the ultrasonic beam.

The peak resonant frequency can be determined either in vivo or in vitro, but preferably in vivo, by exposing the microbubbles to ultrasound, receiving the reflected resonant frequency signals and analyzing the spectrum of signals received to determine the peak, using conventional means. The peak, as so determined, corresponds to the peak resonant frequency (or second harmonic, as it is sometimes termed). The frequency of the sound used may vary from about 0.025 to about 100 megahertz. Frequency ranges between about 0.75 and about 3 megahertz are preferred and frequencies between about 1 and about 2 megahertz are most preferred. Commonly used therapeutic frequencies of about 0.75 to about 1.5 megahertz may be used. Commonly used diagnostic frequencies of about 3 to about 7.5 megahertz may also be used. Ultrasound is generally initiated at lower intensity and duration, and then intensity, time, and/or resonant frequency is increased until the microbubble is visualized on ultrasound (for diagnostic ultrasound applications) or ruptures (for therapeutic ultrasound applications).

Either fixed frequency or modulated frequency ultrasound may be used. Fixed frequency is defined wherein the frequency of the sound wave is constant over time. A modulated frequency is one in which the wave frequency changes over time, for example, from high to low (PRICH) or from low to high (CHIRP). For example, a PRICH pulse with an initial frequency of 10 MHz of sonic energy is swept to 1 MHz with increasing power from 1 to 5 watts. Focused, frequency modulated, high energy ultrasound may increase the rate of local gaseous expansion within the microbubbles and rupturing to provide local delivery of therapeutics. If the microbubbles are produced by microfluidic means as described as one embodiment of this invention, the homogenous nature of the microbubble population will allow efficient bubble rupture and imaging within a narrow frequency range, e.g., 30-40 MHz for Intravascular ultrasound (IVUS), 7-12 MHz for surface vascular ultrasound and 1-3 MHz for clinical echocardiography.

Therapeutic agents may optionally be freed from the microbubbles without rupturing the microbubble. Ultrasound beams that vibrate the microbubbles can allow agents enclosed within the microbubble to pass through the lipid membrane. Agents attached to the lipids may be cleaved from the microbubble surface through enzymatic hydrolysis.

### Detection of Microbubbles

Once administered, microbubbles may be monitored by any suitable means known in the art. In some embodiments, the microbubbles may be monitored and/or detected by ultrasonic imaging means, or by MRI or radiography if the formulation includes agents for such imaging. The ultrasonic irradiation may be carried out by a modified echography probe adapted to simultaneously monitor the reflected echo signal and thereby provide an image of the irradiated site. The monitoring signal may be in the range of 1 MHz to 10 MHz, and preferably between 2 and 7 MHz. In some embodiments, the monitoring signal is in the range of 1-3 MHz.

In some embodiments, the enhanced stability of the PSMs of the present invention would allow visualization of regions further from the administration site and for greater periods of time. Among other assays, PSMs may be used to visualize the vascular system. In visualizing a patient's vasculature, blood flow may be measured, as will be well understood by those skilled in the art. Example of detection methods that can be used in the methods herein are described in US publications U.S. Pat. No. 5,769,080; U.S. Pat. No. 5,209,720; U.S. Pat. No. 6,132,764; U.S. Pat. No. 6,132,764; and US 20100111840, incorporated by reference herein in their entirety.

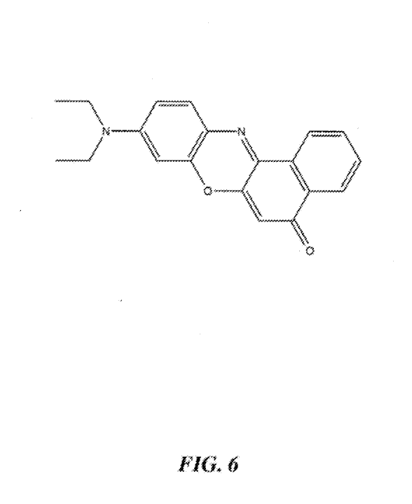

In an exemplary embodiment, following immobilization of the microbubbles, the PAAM-gels were visualized using a portable diagnostic ultrasound system (Terason 2000, Teratech, Burlington, Mass.) along with a 5-10 MHz clinical ultrasound transducer (L10-5, Terason, Burlington, Mass.). The ultrasound setup is shown in FIG. 5. The PAAM-gel was placed at the bottom of a plastic container filled with water. The bottom of the container was composed of an acoustic absorber to prevent reflections. The transducer was then held transfixed through a solid support to a motorized stage with the tip of the transducer in the water. The motorized stage moved the transducer in the x-direction. Starting at one end of the gel, B-mode videos (at least 2 seconds long) were taken approximately every 5 mm in the yz-plane. Videos were saved in audio video interleave (AVI) format and exported for image analysis in MATLAB.

A script was written in MATLAB to take as input an AVI format video and to calculate the average brightness per pixel in each frame. To do this, the script prompts the user to select a region of interest in the gel, and average brightness in that region is calculated in each of the frames of the video. Thus the script outputs an array of brightness values for each frame, essentially calculating the change in brightness over time in the region of interest. A blank gel was used to determine background levels of brightness, and the background was subtracted from the brightness values. The result was an array for each video showing the change in contrast over time provided by the microbubbles in the region of interest, or in other words, the effect of ultrasound insonation on the mechanical stability and echogenicity of the microbubbles.

### Conditions

In some embodiments, the invention provides compositions and methods for the diagnosis and/or treatment of a condition.

In some embodiments, microbubbles may be used with ultrasound, MRI, or other imaging techniques, e.g., to visualize the vasculature, identify and size atheroschlerotic plaques in the vasculature, distinguish between different types of plaques. Ultrasound visualization of microbubbles may also be used to identify and locate solid tumors intravascularly, intranasally, or in other organs, such as intrapulmonary, intrarectal, or intrauterine visualization. PSM usage for therapeutic purposes is potentially limited only by the drugs or other therapeutic agents that can be linked to the microbubbles. As some non-limiting examples, PSMs linked to antiangiogenics may be used to treat tumors, PSMs linked to anti-atherosclerotic drugs may be used to treat plaques in the vasculature, or PSMs linked to local anesthetics may be used to anesthetize a specific area region of interest. Because PSMs can be extremely stable, they may also be administered for use as slow-release capsules to provide a constant, preferably low dosage of a therapeutic agent.

The methods, systems and compositions described herein can be used for the diagnosis and treatment of conditions, e.g. atherosclerosis. Atherosclerosis is the chronic inflammation of the arteries, which through plaque formation and rupture can result in heart attack and stroke. Studies have shown that the vast majority of adults in the United States have atherosclerotic lesions (Tuzcu et al. 2001, Circulation 103, 2705-2710). Current diagnostic techniques concentrate on the size of plaques to determine the risk to the patient. However, plaques vulnerable to rupture differ from stable plaques in molecular composition, not size (Virmani et al. 2006, J. Am. Coll. of Card. 47, C13-18). For this reason, molecular imaging of the cardiovascular system offers a means of identifying vulnerable plaques and would be a substantial improvement over current techniques. Progress has been made in targeted ultrasound contrast imaging, but newer technologies have only resulted in slight increases in acoustic signal in vivo and remain inferior to magnetic resonance imaging (MRI) contrast agents. Thus optimization of targeted ultrasound contrast agents would advance ultrasound's current advantages over MRI of low cost and wide availability. Optimization would likewise improve the safety of contrast imaging by offering control over the size, polydispersity, and stability of particles in the blood stream, advancing these technologies towards FDA approval. Current techniques for synthesizing microbubble contrast agents involve sonication of a liquid mixture in bulk to generate microemulsions, resulting in large polydispersity and variation between batches. Microfluidic flow focusing has the potential to create particles of narrow size distributions. A narrow size distribution is desirable for ultrasound contrast agents because it results in uniform response of the bubbles, greater echogenicity for a population of bubbles, a more selective drug release profile in response to ultrasound, and allows for the optimization of particles to balance ultrasound response and flow characteristics in blood vessels. Likewise, the predictable ultrasound response of monodisperse microbubbles can improve detection by imaging at a specific bandwidth, and also opens the door to quantitative molecular imaging, though these applications are outside the scope of this project.

In addition to applications in imaging, targeted microbubbles could be used in the circulatory system for drug delivery applications. Following angioplasty and/or stenting for the treatment of arterial occlusion, restenosis often occurs, causing the artery to become occluded again. For example, following carotid angioplasty and stenting, the restenosis rate after one year is approximately 6 percent (Groschel et al. 2005, Stroke 36, 367-373). Drug eluting stents have decreased the risk of restenosis, but cause long-term safety concerns, because of potential thrombogenicity and inflammation. Late in-stent thrombosis may be higher in drug-eluting stents, with one report recording four times the incidence relative to bare metal stents after one year (Carlsson et al. 2007, Clin. Res. Cardiol. 96, 86-93). Alternative methods of paclitaxel delivery to sites of inflammation are a current topic of research, in order to prevent the need for the placement of additional stents, which may exacerbate the problem (Herdeg et al. 2008 Thrombosis Res. 123, 236-243; Spargias et al. 2009 J. Intern. Cardiol. 22, 291-298; Unverdorben et al. 2009 Circulation 119, 2986-2994). Microbubbles can be induced to release their contents through ultrasound (Suzuki et al. 2007, J. Control. Release 117, 130-136), offering a means of directing drug release after aggregation of targeted microbubbles at the desired location. This non-invasive method could provide a safer delivery tool to prevent restenosis at damaged sites.

In some embodiments, the invention provides for the imaging of and/or drug delivery to an atheroma. The first imaging technique used to image atheroma was coronary angiography, which uses injection of a contrast agent to reveal blood flow patterns in the patient and vascular tree anatomy. Intravascular ultrasound reveals details about vessel wall thickness, essentially generating a cross-sectional image of the artery through the use of a modified catheter. Though intravascular ultrasound is mainly applied to measure stenosis, limited information about the overall plaque composition can be revealed. For example, intravascular ultrasound can differentiate between calcium-rich plaques and lipid-rich plaques (Nair et al. 2002). Traditional ultrasound is often used, but is limited to vessels close to the skin, such as the carotid arteries, because of limited depth capabilities. Computed tomography (CT) is also used to image atherosclerotic plaques and, like intravascular ultrasound, can differentiate plaques of very different composition. The different imaging techniques have varying strengths and weaknesses that make one preferable over the other in a given situation, such as high dosage of radiation (angiography, CT) or differing anatomical imaging capabilities. However, all of these methods emphasize the degree of stenosis in an artery, revealing little information about the molecular details of the plaque. In order to assess the vulnerability of the plaque, imaging technologies must advance to detect varying levels of molecular markers associated with the different stages of the disease. The targeted microbubbles described herein offer the potential to target specific molecular markers on the atheroma while imaging affected arteries with no radiation dosage. Additionally, in some embodiments, by utilizing microbubbles as drug delivery vehicles, drugs can be targeted and induced to release upon ultrasound stimulation, offering a means of image-guided drug delivery.

In some embodiments, the microbubbles are used for the treatment of an inflammatory condition. For instance, the microbubbles can be used to treat Encephalomyelitis. Further, in other embodiments the microbubbles are used for the treatment of obstructive pulmonary disease. This is a disease state characterized by airflow limitation that is not fully reversible. The airflow limitation is usually both progressive and associated with an abnormal inflammatory response of the lungs to noxious particles or gases. Chronic obstructive pulmonary disease (COPD) is an umbrella term for a group of respiratory tract diseases that are characterized by airflow obstruction or limitation. Conditions included in this umbrella term are: chronic bronchitis, emphysema, and bronchiectasis.

In another embodiment, the microbubbles are used for the treatment of Asthma. Also, the microbubbles are used for the treatment of Endotoxemia and sepsis. In one embodiment, the microbubbles are used to for the treatment of rheumatoid arthritis (RA). In another embodiment, the microbubbles are used for the treatment of Psoriasis. In yet another embodiment, the microbubbles are used for the treatment of contact or atopic dermatitis. Contact dermatitis includes irritant dermatitis, phototoxic dermatitis, allergic dermatitis, photoallergic dermatitis, contact urticaria, systemic contact-type dermatitis and the like. Irritant dermatitis can occur when too much of a substance is used on the skin of when the skin is sensitive to certain substance. Atopic dermatitis, sometimes called eczema, is a kind of dermatitis, an atopic skin disease.

Further, the microbubbles may be used for the treatment of Glomerulonephritis. Additionally, the microbubbles may be used for the treatment of Bursitis, Lupus, Acute disseminated encephalomyelitis (ADEM), Addison's disease, Antiphospholipid antibody syndrome (APS), Aplastic anemia, Autoimmune hepatitis, Coeliac disease, Crohn's disease, Diabetes mellitus (type 1), Goodpasture's syndrome, Graves' disease, Guillain-Barré syndrome (GBS), Hashimoto's disease, inflammatory bowel disease, Lupus erythematosus, Myasthenia gravis, Opsoclonus myoclonus syndrome (OMS), Optic neuritis, Ord's thyroiditis, ostheoarthritis, uveoretinitis, Pemphigus, Polyarthritis, Primary biliary cirrhosis, Reiter's syndrome, Takayasu's arteritis, Temporal arteritis, Warm autoimmune hemolytic anemia, Wegener's granulomatosis, Alopecia universalis, Chagas' disease, Chronic fatigue syndrome, Dysautonomia, Endometriosis, Hidradenitis suppurativa, Interstitial cystitis, Neuromyotonia, Sarcoidosis, Scleroderma, Ulcerative colitis, Vitiligo, Vulvodynia, Appendicitis, Arteritis, Arthritis, Blepharitis, Bronchiolitis, Bronchitis, Cervicitis, Cholangitis, Cholecystitis, Chorioamnionitis, Colitis, Conjunctivitis, Cystitis, Dacryoadenitis, Dermatomyositis, Endocarditis, Endometritis, Enteritis, Enterocolitis, Epicondylitis, Epididymitis, Fasciitis, Fibrositis, Gastritis, Gastroenteritis, Gingivitis, Hepatitis, Hidradenitis, Ileitis, Iritis, Laryngitis, Mastitis, Meningitis, Myelitis, Myocarditis, Myositis, Nephritis, Omphalitis, Oophoritis, Orchitis, Osteitis, Otitis, Pancreatitis, Parotitis, Pericarditis, Peritonitis, Pharyngitis, Pleuritis, Phlebitis, Pneumonitis, Proctitis, Prostatitis, Pyelonephritis, Rhinitis, Salpingitis, Sinusitis, Stomatitis, Synovitis, Tendonitis, Tonsillitis, Uveitis, Vaginitis, Vasculitis, or Vulvitis.

In some embodiments, the microbubbles may be used for the treatment of cancers. In some embodiments, the invention provides a method of treating breast cancer such as a ductal carcinoma in duct tissue in a mammary gland, medullary carcinomas, colloid carcinomas, tubular carcinomas, and inflammatory breast cancer. In some embodiments, the invention provides a method of treating ovarian cancer, including epithelial ovarian tumors such as adenocarcinoma in the ovary and an adenocarcinoma that has migrated from the ovary into the abdominal cavity. In some embodiments, the invention provides a method of treating cervical cancers such as adenocarcinoma in the cervix epithelial including squamous cell carcinoma and adenocarcinomas. Similarly the invention provides methods to treat prostate cancer, such as a prostate cancer selected from the following: an adenocarcinoma or an adenocarinoma that has migrated to the bone. Similarly the invention provides methods of treating pancreatic cancer such as epitheliod carcinoma in the pancreatic duct tissue and an adenocarcinoma in a pancreatic duct. Similarly the invention provides methods of treating bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers. Similarly, the invention provides methods of treating acute myeloid leukemia (AML), preferably acute promyleocytic leukemia in peripheral blood. Similarly the invention provides methods to treat lung cancer such as non-small cell lung cancer (NSCLC), which is divided into squamous cell carcinomas, adenocarcinomas, and large cell undifferentiated carcinomas, and small cell lung cancer. Similarly the invention provides methods to treat skin cancer such as basal cell carcinoma, melanoma, squamous cell carcinoma and actinic keratosis, which is a skin condition that sometimes develops into squamous cell carcinoma. Similarly the invention provides methods to treat eye retinoblastoma. Similarly the invention provides methods to treat intraocular (eye) melanoma. Similarly the invention provides methods to treat primary liver cancer (cancer that begins in the liver). Similarly, the invention provides methods to treat kidney cancer. In another aspect, the invention provides methods to treat thyroid cancer such as papillary, follicular, medullary and anaplastic. Similarly the invention provides methods to treat AIDS-related lymphoma such as diffuse large B-cell lymphoma, B-cell immunoblastic lymphoma and small non-cleaved cell lymphoma. Similarly the invention provides methods to treat Kaposi's sarcoma. Similarly the invention provides methods to treat viral-induced cancers. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. Similarly the invention provides methods to treat central nervous system cancers such as primary brain tumor, which includes gliomas (astrocytoma, anaplastic astrocytoma, or glioblastoma multiforme), Oligodendroglioma, Ependymoma, Meningioma, Lymphoma, Schwannoma, and Medulloblastoma. Similarly the invention provides methods to treat peripheral nervous system (PNS) cancers such as acoustic neuromas and malignant peripheral nerve sheath tumor (MPNST) including neurofibromas and schwannomas. Similarly the invention provides methods to treat oral cavity and oropharyngeal cancer. Similarly the invention provides methods to treat stomach cancer such as lymphomas, gastric stromal tumors, and carcinoid tumors. Similarly the invention provides methods to treat testicular cancer such as germ cell tumors (GCTs), which include seminomas and nonseminomas; and gonadal stromal tumors, which include Leydig cell tumors and Sertoli cell tumors. Similarly the invention provides methods to treat testicular cancer such as thymus cancer, such as to thymomas, thymic carcinomas, Hodgkin disease, non-Hodgkin lymphomas carcinoids or carcinoid tumors.

### Compositions

The present invention is also directed toward therapeutic/diagnostic compositions comprising the therapeutic/diagnostic agents of the present invention. The sizes of the microbubbles may be different for different applications. For general vascular imaging and therapy, sizes may range from about 30 nm to about 10 μm in diameter, preferably between about 2 μm and about 5 μm in diameter. In some embodiments, sizes may range from about 2 μm to about 4 μm. In some embodiments, for applications in tumors or in organs such as the liver, smaller microbubbles (less than 2 μm in diameter) are preferred. Larger microbubbles may be used for imaging or delivery intrarectally or intranasally, up to about 100 μm in diameter.

In some embodiments, the therapeutic delivery systems of the invention are administered in the form of an aqueous suspension such as in water or a saline solution (e.g., phosphate buffered saline). Preferably, the water is sterile. Also, preferably the saline solution is an isotonic saline solution, although, if desired, the saline solution may be hypotonic (e.g., about 0.3 to about 0.5% NaCl). The solution may also be buffered, if desired, to provide a pH range of about pH 5 to about pH 7.4. In addition, dextrose may be preferably included in the media. Further solutions that may be used for administration of PSMs include, but are not limited to almond oil, corn oil, cottonseed oil, ethyl oleate, isopropyl myristate, isopropyl palmitate, mineral oil, myristyl alcohol, octyldodecanol, olive oil, peanut oil, persic oil, sesame oil, soybean oil, and squalene.

Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel™, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

Generally, the therapeutic/diagnostic agents used in the invention are administered to an animal in an effective amount. Generally, an effective amount is an amount effective to (1) reduce the symptoms of the condition sought to be treated, (2) induce a pharmacological change relevant to treating the condition sought to be treated or (3) detect the microbubbles in vivo or in vitro. For cancer, for example, an effective amount includes an amount effective to: reduce the size of a tumor, slow the growth of a tumor; prevent or inhibit metastases; or increase the life expectancy of the affected animal.

Effective amounts of the therapeutic/diagnostic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The therapeutic agents of the instant invention can be administered by any suitable means as described herein, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any animal, preferably to mammals, and more preferably to humans.

The particular mode of administration will depend on the condition to be treated. It is contemplated that administration of the agents of the present invention may be via any bodily fluid, or any target or any tissue accessible through a body fluid.

Preferred routes of administration of the cell-surface targeted therapeutic agents of the present invention are by intravenous, interperitoneal, or subcutaneous injection including administration to veins or the lymphatic system. A targeted agent can be designed to focus on markers present in any fluids, body tissues, and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

As an example of one treatment route of administration through a bodily fluid is one in which the condition to be treated is rheumatoid arthritis. In this embodiment of the invention, the invention provides therapeutic agents to treat inflamed synovia of people afflicted with rheumatoid arthritis. This type of therapeutic agent is a radiation synovectomy agent. The route of administration through the synovia may also be useful in the treatment of osteoarthritis. Delivery of agents by injection of targeted carriers to synovial fluid to reduce inflammation, inhibit degradative enzymes, and decrease pain is envisioned in some embodiments of the invention.

Another route of administration is through ocular fluid. When the vasculature of the eye is targeted, it should be appreciated that targets may be present on either side of the vasculature. Delivery of the agents of the present invention to the tissues of the eye can be in many forms, including intravenous, ophthalmic, and topical. For ophthalmic topical administration, the agents of the present invention can be prepared in the form of aqueous eye drops such as aqueous suspended eye drops, viscous eye drops, gel, aqueous solution, emulsion, ointment, and the like. Additives suitable for the preparation of such formulations are known to those skilled in the art. In the case of a sustained-release delivery system for the eye, the sustained-release delivery system may be placed under the eyelid or injected into the conjunctiva, sclera, retina, optic nerve sheath, or in an intraocular or intraorbitol location. Intravitreal delivery of agents to the eye is also contemplated. Such intravitreal delivery methods are known to those of skill in the art. The delivery may include delivery via a device, such as that described in U.S. Pat. No. 6,251,090 to Avery.

In a further embodiment, the therapeutic agents of the present invention are useful for gene therapy. As used herein, the phrase “gene therapy” refers to the transfer of genetic material (e.g., DNA or RNA) of interest into a host to treat or prevent a genetic or acquired condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Polymerized shell microbubbles of this invention may be stored dry or suspended in a variety of liquid solutions, including distilled water or in aqueous solutions. Aqueous solutions may be buffered to suitable pH ranges (about 5 to about 7.4) by HEPES, Tris, phosphate, acetate, citrate, phosphate, bicarbonate, or other buffers, and may contain isotonic (about 0.9% NaCl) or hypotonic (about 0.3 to about 0.5% NaCl) salt concentrations.

The solutions may also include emulsifying and/or solubilizing agents. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propyleneglycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. Suspending and/or viscosity-increasing agents that may be used with lipid or microbubble solutions include but are not limited to, acacia, agar, alginic acid, aluminum monostearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xanthum gum.

Bacteriostatic agents may also be included with the microbubbles to prevent bacterial degradation on storage. Suitable bacteriostatic agents include but are not limited to benzalkonium chloride, benzethonium chloride, benzoic acid, benzyl alcohol, butylparaben, cetylpyridinium chloride, chlorobutanol, chlorocresol, methylparaben, phenol, potassium benzoate, potassium sorbate, sodium benzoate and sorbic acid.

### Administration

The methods involve the administration of one or more microbubbles, e.g., for the diagnosis and/or treatment of a condition. In some embodiments, other agents are also administered, e.g., other therapeutic agent. When two or more agents are co-administered, they may be co-administered in any suitable manner, e.g., as separate compositions, in the same composition, by the same or by different routes of administration.

The microbubbles of this invention may be administered in a variety of methods, such as intravascularly, intralymphatically, parenterally, subcutaneously, intramuscularly, intranasally, intrarectally, intraperitoneally, interstitially, into the airways via nebulizer, hyperbarically, orally, topically, or intratumorly, using a variety of dosage forms. In some embodiments, the microbubbles are injected intravenously. In some embodiments, the microbubbles are injected intraarterially. The microbubbles may also be utilized in vitro, such as may be useful for diagnosis using tissue biopsies.

In some embodiments, the microbubbles are administered in a single dose, e.g, for the treatment of an acute condition. Typically, such administration will be by injection. However, other routes may be used as appropriate. In some embodiments, the microbubbles are administered in multiple doses. Dosing may be about once, twice, three times, four times, five times, six times, or more than six times per day. Dosing may be about once a month, once every two weeks, once a week, or once every other day. In one embodiment the microbubbles are administered about once per day to about 6 times per day. In another embodiment the administration of the microbubbles continue for less than about 7 days. In yet another embodiment the administration continues for more than about 6, 10, 14, 28 days, two months, six months, or one year. In some cases, continuous dosing is achieved and maintained as long as necessary. In some embodiments, the microbubbles are administered continually or in a pulsatile manner, e.g. with a minipump, patch or stent.

Administration of the microbubbles of the invention may continue as long as necessary. In some embodiments, an agent of the invention is administered for more than 1, 2, 3, 4, 5, 6, 7, 14, 28 days or 1 year. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

When diagnosis and/or treatment need to be performed as a series, e.g., a series of diagnostic tests after treatment, the diagnosis and/or treatment may be performed at fixed intervals, at intervals determined by the status of the most recent diagnostic test or tests or by other characteristics of the individual, or some combination thereof. For example, diagnosis and/or treatment may be performed at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for diagnosis and/or treatment and the availability of diagnostic/treatment facilities, thus approximate intervals corresponding to an intended interval scheme are encompassed by the invention. As an example, an individual who has undergone treatment for a cancer may be tested/treated relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, intervals may be modified.

In one embodiment, a diagnostic test may be performed on an apparently healthy individual during a routine checkup and analyzed so as to provide an assessment of the individual's general health status. In another embodiment, a diagnostic test may be performed to screen for commonly occurring diseases. Such screening may encompass testing for a single disease, a family of related diseases or a general screening for multiple, unrelated diseases. Screening can be performed weekly, bi-weekly, monthly, bi-monthly, every several months, annually, or in several year intervals and may replace or complement existing screening modalities.

Progression in the circulation of the administered microbubble formulation toward the selected site may be monitored any suitable method known in the art, including those described herein, e.g., by ultrasonic imaging means, or by MRI or radiography if the formulation includes agents for such imaging. In some embodiments, the circulation of the administered microbubble formulation toward the selected site is monitored using ultrasonic imaging means. The ultrasonic irradiation may be carried out by a modified echography probe adapted to simultaneously monitor the reflected echo signal and thereby provide an image of the irradiated site. The monitoring signal can be in the range of 1 MHz to 10 MHz and preferably between 2 and 7 MHz.

The useful dosage of gas-filled microbubbles to be administered and the mode of administration will vary depending upon the age, weight, and mammal to be treated, and the particular application (therapeutic/diagnostic) intended. Typically, dosage is initiated at lower levels and increased until the desired therapeutic effect or imaging visibility is achieved.

### Kits

The invention also provides kits. The kits include the microbubbles described herein, in suitable packaging, and written material that can include instructions for use, discussion of clinical studies, listing of side effects, and the like. Suitable packaging and additional articles for use (e.g., measuring cup for liquid preparations, foil wrapping to minimize exposure to air, and the like) are known in the art and may be included in the kit.

The microbubbles may be provided dry or in a storage solution, and may be pre-polymerized or polymerized before administration, e.g. by UV light exposure. The microbubbles solutions may be ready for administration immediately, or may be suspended or mixed with additional compounds or solutions before administration. The microbubbles provided may already contain therapeutic or contrast agents for usage, or such agents may be linked or incorporated into the microbubbles on-site. Microbubbles may further be provided in specific sizes for different routes of administration or for response to specific ultrasound frequencies, or may be comprised of a heterogeneous distribution of sizes.

The reagents may also include ancillary agents such as buffering agents and stabilizing agents, e.g., polysaccharides and the like. The kit may further include, where necessary, agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of the microbubbles, e.g. ultrasound imaging, which are suitable for the clinical detection, prognosis, and screening of cells and tissue from patients, such as the conditions described herein.

Such kits may additionally comprise one or more therapeutic agents. The kit may further comprise a software package for data analysis, which may include reference date for comparison with the test results.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

This invention is further illustrated by the following examples which should not be construed as limiting. The contents of all references cited throughout this application, as well as the figures and table are incorporated herein by reference.

### EXAMPLES

### Example 1

### Preparation of a Flow Focusing Microfluidic Device and it Use to Synthesize Uniformly Sized Polymerized Shell Microbubbles

The goal of this example was to prepare a flow focusing microfluidic device and use it to synthesize uniformly sized polymerized shell microbubbles. The resultant microbubbles were characterized according to size and polydispersity, and the lipid shell was successfully polymerized. Microbubbles were fabricated using flow focusing devices capable of single emulsions to generate the polymerized lipid shelled microbubbles, or polymerized shell microbubbles (PSMs). Following on-chip fabrication, PSMs with varying mole fraction of polymerizable lipid were polymerized using UV light. Microbubbles were then characterized according to size and polydispersity to demonstrate successful fabrication. Additionally, polymerization of the lipid shell was confirmed through spectrophotometric studies.

### Design and Methods

The following methods were used to synthesize PSMs through the flow focusing microfluidic technique to produce microbubbles that had a narrow polydispersity and controlled size, between 3-5 μm in diameter.

In order to synthesize uniformly sized gas bubbles with a polymerized lipid shell, it was necessary to fabricate a flow focusing microfluidic device. The development of these devices, from design through fabrication, is described in the following sections along with the procedures to form the lipid mixtures. The overarching design goals are shown in FIG. 2. Following completion of the microfluidic device and preparation of the polymerizable lipid mixtures, the polymerized shell microbubbles were fabricated.

In addition to the fabrication of the microbubbles, the methods used to characterize the resulting product are described in detail herein.

### Description of Device

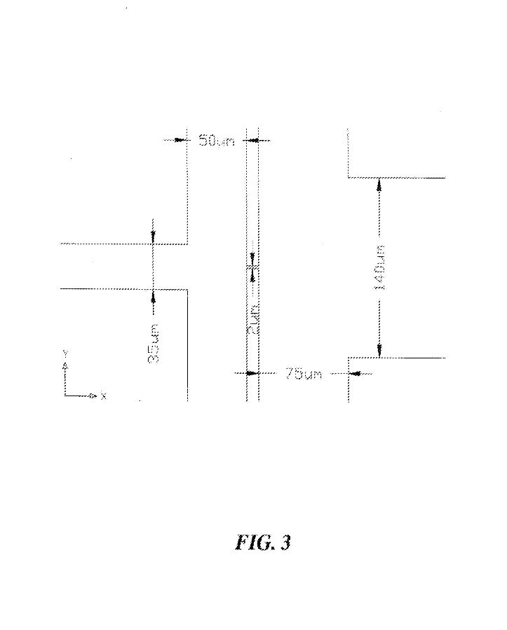

A single emulsion microfluidic device was designed using AUTOCAD. The design was modified and scaled down from a previously described microfluidic flow focusing device (Anna et al. 2003, Appl. Phys. Lett. 82, 364-366). The layout of the device orifice, where the emulsification occurs, is shown in FIG. 3. The gas enters the device through the 35 μm channel, and is focused through the orifice by the lipid solution, which flows through the 50 μm channels. This results in the pinching off of bubbles of gas, which are quickly stabilized by the formation of a monolayer of lipids at the gas-water interface, and exit through the 140 μm channel. The 75 μm channel could be used to form a second emulsion, or in this case was used to clear debris or clogs when they occurred. The 35 μm channel carries the decafluorobutane gas to the 2 μm orifice, where it is focused by the lipid solution streams from the 50 μm channels. The resultant microbubbles exit through the 140 μm channel.

The process of advancing from an AUTOCAD design to an actual microfluidic device involved several steps. The basic steps that were followed are shown in FIG. 4. This flowchart describes the process of fabrication from design to finished device. Following design of the device, a photomask of the design must be fabricated in order to perform photolithography of the design. Next, using the photomask, photolithography techniques were used to pattern the design onto photoresist applied on a silicon wafer. This photoresist was baked and surface treated to allow it to be used as a mold for the elastomeric polydimethylsiloxane (PDMS), the material used for the microfluidic devices. The PDMS was then poured and baked onto the micropatterned wafer. Finally, the PDMS was removed, cut into individual devices, hole-punched for portholes, and bonded to glass cover slips. The result was a completed microfluidic device ready for use.

### Mask Writing

The AUTOCAD device design was used to write a mask using a DWL66 mask writer (Heidelberg Instruments, Heidelberg, Germany). The DWL66 software required the AUTOCAD file (.dxf) to be converted using a computer connected to the DWL66 in the Photonics Center class 100 cleanroom. Following the file conversion, the file was transferred to the mask writer. A 10 mm write head was used, resulting in resolution of 1 μm. A resist-coated chrome mask was loaded into the mask writer (Nanofilm, West Lake, Calif.) and held in place through a vacuum seal. The parameters for use were continually updated by cleanroom staff through weekly calibrations, the parameters dictated for the specified write head were selected, and the mask writing was initiated. Following an approximately 5 hour processing time, the mask was removed and developed for 2 minutes using AZ300MIF developer (AZ Electronic Materials, Somerville, Mass.). At this point the design was visible in the developed photoresist. Following development, the mask was placed in chromium etchant 1020 (Transene Company Inc., Danvers, Mass.) for 2 minutes, after which the portion of the mask with the design was transparent, while the remaining area remained opaque. Subsequently, the remaining resist was stripped using a multi step process to ensure that no residual resist compromised the mask functionality. The mask was placed in 1165 resist stripper (Rohm Haas, Marlborough, Mass.) for at least 30 minutes at 70° C. Next, the wafer was rinsed with water to remove the resist stripper and residual resist. Following the rinse, acetone was used along with non-abrasive cotton swabs to scrub the mask clean, especially in areas with thin walls. The device was then observed under a microscope to check for residual resist, and the acetone cleaning process was repeated as much as necessary. Finally, the mask was rinsed with acetone, methanol, and isopropanol and dried with nitrogen gas.

### SU8 Photolithography

Photolithography was used to produce SU8 molds of the device on a silicon wafer, later used to imprint the design on PDMS stamps. A 10 cm silicon wafer (University Wafers, Boston, Mass.) was cleaned using subsequent acetone, methanol and isopropanol rinsing followed by nitrogen gas to remove remaining solvent. The wafer was then dehydrated on a hotplate at 95° C. for 5 minutes. Finally, the wafer was cleaned in an MIA Plasma Asher (PVA TePla, Corona, Calif.) for 5 minutes using high oxygen flow. Following the cleaning steps, the wafer was placed on a Delta 80 RC/T3 Spinner (Suss, Garching, Germany) and SU8-2005 (MicroChem, Newton, Mass.) was spun and patterned onto the wafer with a thickness of 10 μm using the following steps: (i) Apply SU8 2005 onto wafer, (ii) Spin 1000 rpm for 2 minutes, (iii) Soft bake at 65° C. for 1 minute, 95° C. for 2 minutes, and 65° C. for 1 minute, (iv) Expose in MA6 Mask Aligner (Suss Microtec, Garching, Germany) for 1 minute on CH2 using soft contact exposure, and (v) Develop for 1 minute in SU8 developer (MicroChem, Newton, Mass.)

The completed wafer was then rinsed with DI water and dried with nitrogen gas.

### Preparation of PDMS Devices

In order to make PDMS stamps from the SU8 molds, it was necessary to first coat the molds with silane. The silicon wafer containing the molds was placed at the bottom of a Petri dish, while 10 μL of (heptafluoropropyl)trimethylsilane, 97% (Sigma, St. Louis, Mo.) was placed in the dish next to the wafer. The entire setup was then placed under vacuum overnight, resulting in a well-coated wafer.

With the preparation of the SU8 molds complete, the next steps were to mix the PDMS, pour it over the wafer in the Petri dish, and bake it. Sylgard 184 PDMS (Dow Corning, Midland, Mich.) was prepared by mixing monomer and curing agent at the dictated ratio of 10 to 1. Sufficient PDMS was prepared to produce devices of approximately 1 cm thickness, resulting in the production of approximately 70 g of PDMS. The mixed solution was poured over the wafer in the Petri dish and placed in a vacuum chamber to degas for 2 hours. After this degassing period, any remaining bubbles were popped using a needle. Finally, the entire setup was placed in an oven at 80° C. for at least 2 hours to cure the PDMS.

After removal from the oven, the dish was allowed to cool for at least 20 minutes. The next steps were done in a horizontal laminar flow cabinet (NuAire, Plymouth, Minn.) to limit dust exposure of the devices. Next, the mold of the wafer was cut out of the dish, and individual devices were separated using a scalpel. The portholes were then made using a 0.75 mm diameter Harris Uni-Core hole-punch (Sigma, St. Louis, Mo.).

The devices were then immediately taken to the cleanroom to avoid dust accumulation. There the PDMS stamps were washed using acetone, methanol, and isopropanol, and dried with nitrogen gas. They were then placed in the an MIA Plasma Asher (PVA TePla, Corona, Calif.), along with one glass cover slip (3 inches by 2 inches) per stamp, for 5 minutes using high oxygen flow Immediately following the plasma treatment, the PDMS stamps and glass slides were removed from the machine and each stamp was placed, design side down, on a glass slide. Light pressure was applied until bonding was observed, which was typically either immediate or occurred within a few seconds. The bonded devices were then placed in Petri dishes and baked overnight at 80° C. to ensure a strong bond.

### Preparation of Lipids

Lipids in powder form were dissolved in chloroform and combined at the desired mole fractions. Lipid mixtures were a combination of commercially available lipids (Avanti, Alabaster, Ala.) and proprietary polymerizable lipids obtained from NanoValent Pharmaceuticals. Upon combination, the mixtures were vortexed for several seconds to fully mix the lipids. The chloroform mixture was then placed in a vacuum oven at 45° C. until the solvent evaporated. The lipids were then placed in vacuum for at least 2 additional hours at room temperature to completely remove the chloroform. The lipid powder was then dissolved in sterile filtered 10% glycerol, 10% propylene glycol, 80% DI water (10:10:80 solution). The lipid mixtures were dissolved in the 10:10:80 aqueous solution at a concentration of 5.32 μmol/mL. The lipid solution was then heated to 60° C. and placed in a sonication bath for at least 30 minutes, until the solution became clear. The different lipid mixtures prepared are described in Table 1.

TABLE 1
Mole fractions of lipid mixtures.

| | Specific Lipid Components | | | | Totals | |
|---|---|---|---|---|---|---|
| | Diacetylenic- | Diacetylenic- | | | Polymerizable | |
| | PEG1 | PEG2000 | Soy-PC | PE-PEG2000 | Lipid | PEG2000 |
| PSMO | 0% | 0% | 85% | 15% | 0% | 15% |
| PSM25 | 10% | 15% | 75% | 0% | 25% | 15% |
| PSM50 | 35% | 15% | 50% | 0% | 50% | 15% |

The diacetylene containing lipids are proprietary and were obtained from NanoValent Pharmaceuticals, while the Soy-PC (L-α-phosphatidylcholine) and PE-PEG2000 (1,2-distearoyl-sn-glycero-3-phosphoethanolamine-N-[methoxy(polyethylene glycol)-2000]) were obtained commercially from Avanti Polar Lipids.

Additionally, for the conjugation experiment the PSMO formulation was modified by replacing ⅓ of the PE-PEG2000 (5% of total) with PE-PEG2000-biotin.

Prior to making microbubbles using the lipid solutions, it was necessary to saturate them with the gas used to make the bubbles, decafluorobutane. To this end, each lipid solution was placed under vacuum overnight to extricate air from the solution. Next, lipids were placed under decafluorobutane gas at a pressure of 5PSI, and stirred for at least one hour, resulting in saturation with the gas.

### Microfluidic Production of Microbubbles

PDMS microfluidic devices were plasma treated in a Harrkick PDC-32G Plasma Cleaner (Hulick, Ithaca, N.Y.) for 5 minutes in order to make the surfaces hydrophilic to facilitate complete wetting of the interior of the devices. Polyethylene tubing (Becton Dickinson, Franklin Lakes, N.J.) was then inserted into the input portholes. Lipid solution was drawn into a 1 mL syringe, which was attached to the device in series with a 4 mm, 0.2 μm pore size syringe filter (Corning, Corning, N.Y.), a 23 gauge dispensing needle (McMaster-Carr, Elmhurst, Ill.), and tubing. Decafluorobutane gas (Synquest Laboratories, Alachua, Fla.) was attached to the device in a pressure-controlled manner. The canister was attached to a pressure regulator (Swagelok, Solon, Ohio) followed by a needle valve and a pressure meter. Finally, the gas was passed through a 0.41 m syringe filter and into the device via tubing.

Lipid solution was pumped into the device using a PHD2000 Harvard Apparatus syringe pump (Harvard Apparatus, Holliston, Mass.). Flow rate was controlled, and was varied in order to obtain microbubbles of different sizes, from approximately 1 μL/min up to 10 μL/min Likewise, gas pressure was controlled using the pressure regulator, again varied to control the size and distribution of microbubbles produced, from 4PSI to 10 PSI. When the lipid solution was close to entering the device, the gas valve was opened to allow the gas to enter the device.

Microbubble production was monitored using an Axiovert 25 microscope (Zeiss, Oberkochen, Germany) and recorded with a high-speed camera (Photron, San Diego, Calif.). The solution containing microbubbles was collected at the output port. To visualize microbubbles through microscopy after collection, droplets of microbubble solution were placed between glass slides separated by approximately 3 mm, and images of microbubbles accumulated on the top slide were taken.

### Polymerization of Microbubble Shells

Collected microbubbles in aqueous solution were distributed in wells of a 96 well plate, and dispersed with a pipette prior to UV treatment. The plate was placed 6 inches directly under a germicidal 30W T8 UV lamp (General Electric, Fairfield, Conn.) and subjected to either 2 minutes or 5 minutes of UV light.

### Size and Distribution Characterization

A Coulter Z2 Analyzer (Beckman Coulter, Brea, Calif.) was used to characterize the size and distribution of particles. Following fabrication of microbubbles or PSMs, up to 5004, of microbubble aqueous solution was diluted in 15 mL of Isoton (Beckman Coulter, Brea, Calif.). The solution was then analyzed in the Coulter Z2 using a 0.5 mL analytical volume and measuring microbubbles with diameters from 3 to 9 μm.

The lower limit of the Coulter Z2 Analyzer for particle sizing is 2 μm diameter microbubbles. As the particle distribution approaches this lower limit, the level of noise becomes excessive and masks the actual distribution. For this reason, it was important to keep microbubble distributions well above this lower limit in order to get clean data. In order to assess the microbubble solutions for the presence of smaller particles, or to characterize populations below the noise threshold for the Coulter Z2 Analyzer, dynamic light (DLS) scattering was used. The Brookhaven 90Plus Particle Size Analyzer (Brookhaven, Holtsville, N.Y.) can size particles from 1 nm up to a few microns, though experience has shown that particles larger than 2 microns result in unreliable data acquisition due to the method of sizing (dynamic light scattering).

To run the 90Plus, 100 μL of microbubble solution was diluted in 3 mL DI water to fill a cuvette. The standard operating procedures of the Biointerface Technologies Core at Boston University were followed. The average count rate was measured and the solution was either diluted or concentrated if necessary to get the average count rate in the 50-300 Kcps range. Three runs of 1 minute were done for each sample and the data were averaged together and exported for later analysis.

### Absorbance Spectrum and Fluorescence Comparison

A characteristic color change is associated with the polymerization of diacetylenic lipids (Johnston et al. 1980, Biochim. Biophys. Acta 602, 57-69). This change was easily visualized macroscopically, as the solution turned from clear to blue or red (depending on the state of the lipid). The red state is also fluorescent. To confirm polymerization experimentally, an absorbance spectrum was obtained from a diluted PSM solution, diluted standard lipid microbubble solution, and the aqueous solution without microbubbles or lipids.

To this end, microbubbles were fabricated as described previously and PSMs were UV treated for 5 minutes. Both lipid solutions were diluted Ito 100 in the 10:10:80 aqueous solution and loaded into a SpectraMax M5 plate reader (Molecular Devices, Sunnyvale, Calif.) along with a sample of pure 10:10:80 solution. An absorbance spectrum from 400-700 nm was obtained and exported for analysis. Additionally, fluorescence was measured on the same machine to further illuminate the state of lipids present. The same samples were excited at 544 nm and emission was measured at 640 nm. Again, data were saved and exported for later analysis.

### Results

Microbubbles were successfully fabricated using the PDMS flow focusing devices at various flow rates and pressures to obtain different sizes and distributions. Four different regimes of microbubble production are shown in FIG. 7A-D. In these pictures microbubbles are seen being formed in the devices and traveling from the orifice, where the gas and lipid solution form the emulsion, out through the output channel. This figure demonstrates the variety of sizes of microbubbles that can be obtained using this method and from the same device by varying the flow rate and gas pressure.

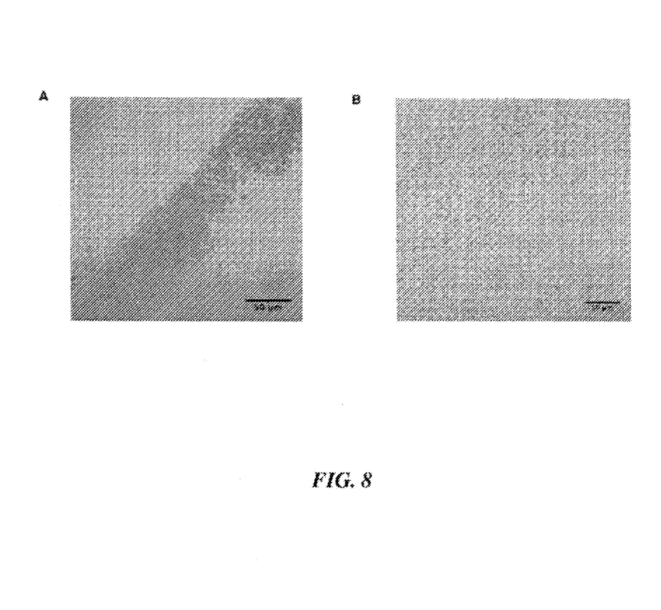

Following production in the device, the microbubbles solution exited through the output port at the top of the device. FIG. 8 shows the microbubbles exiting the device after formation. In FIG. 8A, microbubbles can be seen streaming out from the output port and rising in solution, showing their continued presence following production and exit from the device. Likewise, in FIG. 8B, microbubbles of a slightly smaller size can be seen floating in solution on the top of the device following their exit. It is at this point that the microbubbles are typically gathered via pipette for characterization and further analysis.

### Imaging and Sizing of Microbubbles

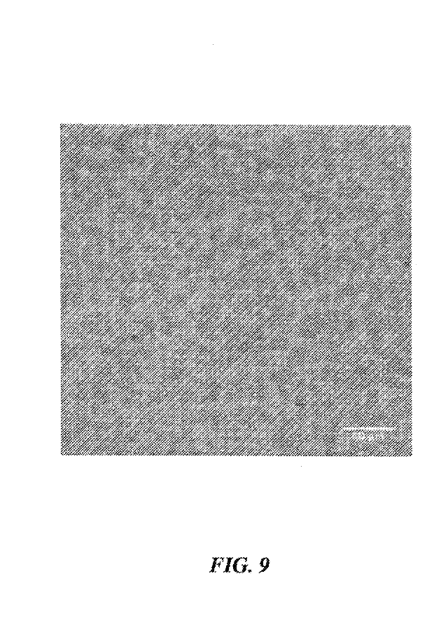

Microbubbles were successfully imaged using phase-contrast microscopy. It was determined that microscopy was not a reliable method for sizing, as microbubbles appeared different sizes on different microscopes and magnifications, due to the fundamental capabilities of phase contrast microscopy. However, it is a convenient way to confirm the presence of microbubbles, to gain valuable information about their distribution, and to get basic information about size. FIG. 9 shows microbubbles floating on a glass slide, collected immediately after production in the flow focusing device. Microbubble solution was captured between glass slides, and this phase contrast image shows monodisperse microbubbles collecting on the top slide.

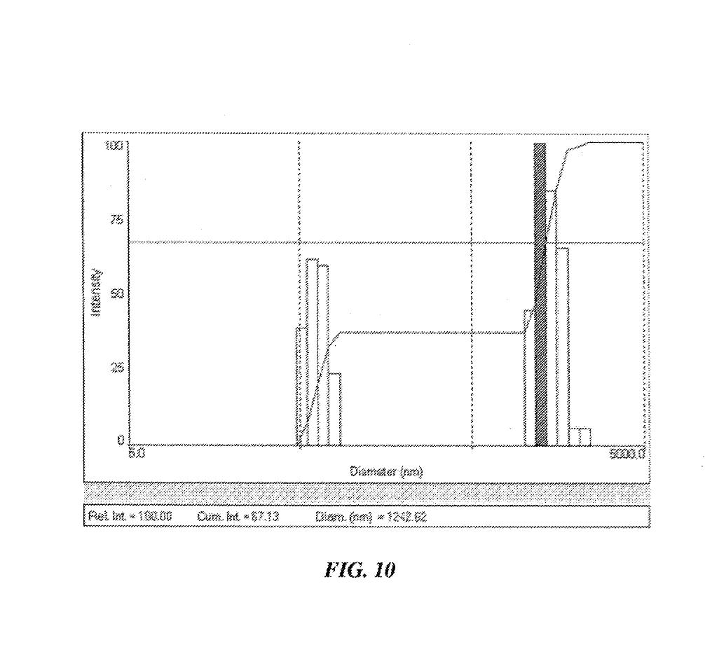

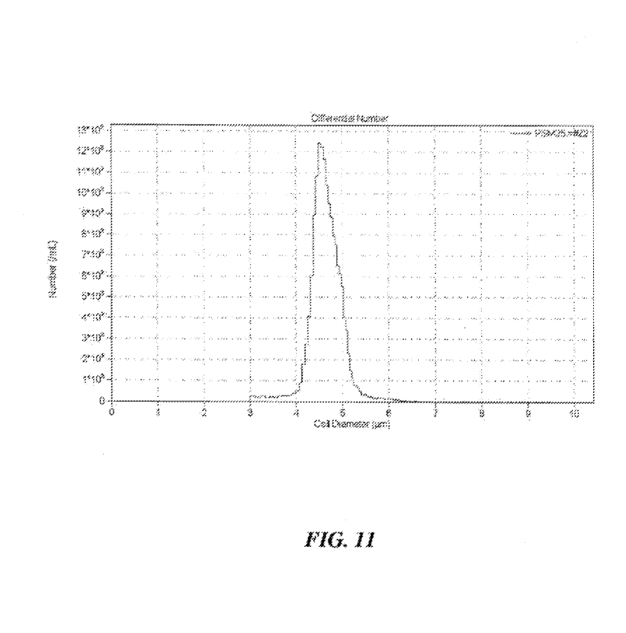

It was clear that the microbubbles formed a relatively monodisperse population, and were present in plentiful quantities. These microbubbles were stable on the glass slide for at least several days. Using basic image analysis techniques, the microbubbles appeared to have an average diameter of approximately 5 μm. However, in the Coulter Z2 Analyzer this population appeared only as noise, indicating an average diameter near or below 2 μm. As a result, dynamic light scattering was used to assess the population. The results are shown in FIG. 10. The smaller population consisted of liposomes formed as a side product of the procedure, while the larger population was the microbubbles seen in the phase contrast image (FIG. 9). The highest intensity peak is highlighted, centered at 1243 nm.

The MS showed two populations of particles, one between 50-200 nm and another centered about 1.2 μm in diameter. The 50-200 nm population was produced during the preparation process, when the lipid solution was first bath sonicated and then filtered using 200 nm pores. This resulted in the production of liquid-filled liposomes, and their extrusion through a membrane produced liposomes of this size. This was a necessary by-product of the process, which could be separated out later if necessary. The larger population in the graph included the microbubbles seen using light microscopy. This population, centered around 1.2 μm in diameter, appeared much larger in the phase contrast image. From these results, it was apparent that the size needed to be increased in order to enter the 3-5 μm range that is ideal for ultrasound contrast.

To increase the size of the microbubbles produced, the lipid solution flow rate and gas pressure were altered. Microbubbles of a suitable distribution were produced at 7 μL/min and 8PSI. The distribution produced was characterized using the Coulter Z2 Analyzer, and the resulting data are shown in FIG. 11. The mean diameter was 4.7 μm, with a standard deviation of 0.42 μm. It can be seen that the signal to noise ratio was satisfactory, with low levels of noise relative to the microbubble population. Coulter Z2 Analyzer data showed a relatively monodisperse population of microbubbles with a mean diameter of 4.7 μm. The polydispersity of the sample of microbubbles was about ±10% of the average microbubbles size in the sample. This represented a significant reduction in the polydispersity of mircobubbles made with non-polymerized lipids.

This microbubble population was well suited for ultrasound contrast applications, balancing the size requirements of navigating capillaries and producing the maximum possible contrast. With this in mind, the size and distribution were sufficiently developed to proceed with further characterization of the microbubbles.

### Absorbance Spectrum and Fluorescence Analysis

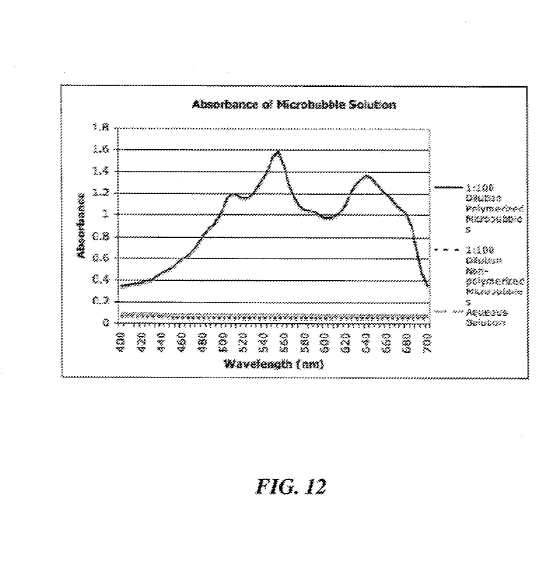

After microbubbles were successfully produced, polymerizable lipid microbubbles were UV treated for 5 minutes, and absorbance spectra were taken to determine if the characteristic color change associated with diacetylene polymerization took place. Microbubble solutions were diluted 1 to 100 in aqueous 10:10:80 solution. FIG. 12 shows the absorbance spectra for polymerized lipid microbubbles, standard lipid microbubbles, and pure 10:10:80 solution. Increased absorbance occurred for the polymerized lipid microbubbles throughout the spectrum, especially between 500 nm and 680 nm with peaks at approximately 560 nm and 645 nm. This figure shows the absorbance spectra of three different solutions: diluted standard lipid microbubbles, diluted polymerized lipid microbubbles, and 10:10:80 aqueous solvent.

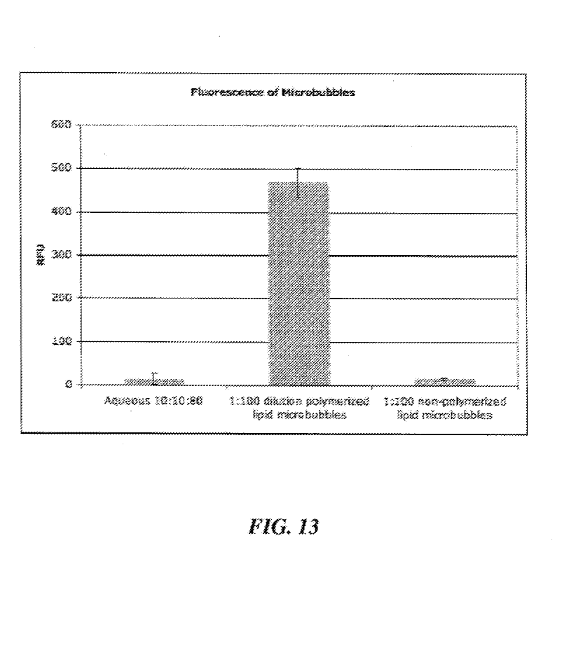

Following the absorbance spectrum analysis, fluorescence was measured for the same solutions, as one of the polymerized lipid states is fluorescent. Two samples were measured for each solution. Solutions were excited at 544 nm and emission was quantified at 640 nm. The data are presented in FIG. 13. Polymerized lipid microbubbles clearly had the most fluorescence, with an intensity of 469 RFU (SD 33.4), while standard lipid microbubbles had a fluorescent intensity of 15.7 RFU (SD 2.52), and the 10:10:80 aqueous solution had an intensity of 14.4 RFU (SD 12.5). Fluorescence of polymerized and non-polymerized microbubbles were compared to the 10:10:80 aqueous solution, with an excitation of 544 nm and emission measured at 640 nm. Note the significant increase associated with UV treatment.

### Discussion

This example describes how to design and fabricate a microfluidic flow focusing system, and demonstrates the system's capability to produce lipid microbubbles of controlled size and distribution. With regard to clinical applications, it is possible to produce microbubbles of an ideal size for ultrasound imaging (diameter of 2-5 μm) and drug delivery among other applications, and the resulting size distribution was characterized using particle sizing methods.

This example introduced the use of polymerizable lipids to produce microbubbles for ultrasound contrast agents. By using lipids that polymerize in response to UV light, microbubbles could be produced using the non-polymerized lipids and polymerization of the microbubble following production induced through UV exposure. Upon polymerization, diacetylenic lipids undergo a characteristic color change to a blue state or a fluorescent red state. To confirm polymerization, spectrophotometric studies were done. After 2 minutes of UV exposure, the microbubble solution went from clear to a purple color. Additionally, the typically white foam on the surface of the solution that is dense with microbubbles turned red. These results were confirmed through absorbance spectra, showing a vast increase in absorbance throughout the visual spectrum with dips at 400-500 nm and 680-700 nm, corresponding to blue and red. Likewise, fluorescent studies showed a 30-fold increase in red fluorescence (excitation 544 nm, emission 640 nm) following polymerization. These results indicate that both blue and red forms of the diacetylenic lipids were present. Previous studies have demonstrated the formation of polymerized liposomes (liquid filled, nanoscale), which are of the blue form until placed under certain stimuli, such as increased heat or extreme pH, that result in the red form (Kauffman et al. 2009, ACS Appl. Mater. & Interfaces 1, 1287-1291). Additionally, previous studies have looked at the relationship between lipid monolayer structure and color through Langmuir films. One such study describes the difference between the metastable blue state and stable red state using diffraction techniques and concludes that the two states have different packing arrangements and densities, with the red phase containing more densely packed lipids (Lifshitz et al. 2009, Langmuir 25, 4469-44). The dynamic light scattering studies described here indicated that a population of liposomes was being produced during lipid solution preparation and filtration, resulting in the population of particles from 50-200 nm in diameter. These results, combined with the red foam observed upon polymerization and previous studies describing blue diacetylenic liposomes, indicated that the microbubbles in solution contain the red phase lipids, and the liposomes in solution contain lipids in the blue phase.

This example successfully demonstrated fabrication of a flow focusing microfluidic device, production of microbubbles of the desired size and distribution, and polymerization of the lipid shell to form a polymerized shell microbubble.

### Example 2

### Characterization of Ultrasound Response and Stability of the Microbubbles

This example characterized the effects of the polymerized lipid shell and shell composition on ultrasound response and stability of the microbubbles. Further studies were performed to demonstrate echogenicity, ultrasound destruction capabilities, and the advantages of the PSM system.

Microbubbles were immobilized in a gel and subjected to ultrasound imaging in order to demonstrate echogenicity and to compare the mechanical stability of the PSMs and standard lipid microbubbles. Additionally, the dissolution of microbubbles of varying composition in a salt solution was characterized.

### Design and Methods

The following methods tested whether microbubbles produced through the described system were echogenic, and whether polymerized lipids exert an effect on mechanical stability. Additionally, the methods also tested whether microbubble stability in a salt solution can be tuned through lipid composition.

### Dissolution Study Protocol

The dissolution study used the same setup as the basic size distribution characterization described in Example 1. However, following the first analysis, the Isoton/microbubble solution was analyzed every 30 minutes for 2 hours, and data were recorded and exported for later analysis. The three lipid formulations were compared. The PSM25 and PSM50 formulations were UV treated for 2 minutes prior to size distribution measurements.

After population distribution information was obtained, the data were analyzed by summing the microbubble counts in the window of ±10% of the population median diameter (at time zero) for each time point. The counts at each time point in this window were then divided by the initial number of counts, resulting in a description of the proportion of remaining microbubbles as time progressed.

### Ultrasound Characterization

Microbubbles were immobilized in a gel and observed under ultrasound using clinical ultrasound technology to determine echogenicity. Additionally, microbubbles were placed under continuous ultrasound to determine differential stability of PSMs relative to standard microbubbles.

### Preparation of PAAM-Gel

Microbubbles were immobilized in a polyacrylamide (PAAM) gel for ultrasound visualization. This allowed for the microbubbles to be visualized in a stationary position. PAAM was selected as the substrate because it has similar acoustic properties to tissue.

In order to fabricate the gel, it was necessary to have a mold to pour the solution into to form a gel of an appropriate size for the ultrasound transducer. To this end, PDMS was poured into a 10 cm Petri dish and cured as described previously. An approximately 1 inch by 1.5 inches section was cut out to produce a 10 mL hole in the mold. To make the gel, it was necessary to have an airtight seal on either side of the PDMS mold. To begin, 2 glass cover slips (3 inches by 2 inches) were coated with commercially available Rain-X and allowed to dry. A glass slide was then bonded to the bottom of the PDMS mold by applying light pressure until bonding was observed. The top slide would be bonded later, once the gel solution was in the mold. Next, 1 mL of 10% ammonium persulfate was prepared by dissolving 100 mg of ammonium persulfate in 1 mL of DI water. The solution was then vortexed until the ammonium perfsulfate was completely dissolved, resulting in a clear solution. To make a 10% polyacrylamide solution 2.5 mL of 40% acrylamide solution (Bio-Rad, Hercules, Calif.) was combined with 2 mL of 2% Bis solution (Bio-Rad, Hercules, Calif.), 50 μL of 10% ammonium persulfate, and 5.5 mL of DI water. Next, up to 1 mL of dilute microbubble solution was added, and the mixture was vortexed for 5 seconds to disperse the microbubbles. Finally, 20 μL, of the crosslinker tetramethylethylenediamine (TEMED) was added and the solution was vortexed for an additional 5 seconds, The gel solution was then poured into the PDMS mold, allowing for the creation of a convex meniscus on the top. A glass slide was then placed on top of the mold, ensuring no bubble formation, and pressed down to form a bond. Once the glass slide was bonded to the top, polymerization of the gel began. During this period, the gel was inverted every 2 minutes in order to prevent accumulation of the bubbles at the top of the gel. After approximately 15 minutes, the gel was removed from the mold, placed in a Petri dish, and hydrated with water. Three gels were compared: one contained PSMO microbubbles, another contained PSM50 microbubbles, and one was absent of microbubbles.

### Ultrasound Procedure

Following immobilization of the microbubbles, the PAAM-gels were visualized using a portable diagnostic ultrasound system (Terason 2000, Teratech, Burlington, Mass.) along with a 5-10 MHz clinical ultrasound transducer (L10-5, Terason, Burlington, Mass.). The ultrasound setup is shown in FIG. 5. The microbubbles were immobilized in a PAAM-gel and placed at the bottom of a non-reflecting container filled with DI water. The ultrasound transducer had freedom to move in the x-direction, while the B-mode ultrasound images were taken in the yz-plane.

The PAAM-gel was placed at the bottom of a plastic container filled with water. The bottom of the container was composed of an acoustic absorber to prevent reflections. The transducer was then held transfixed through a solid support to a motorized stage with the tip of the transducer in the water. The motorized stage moved the transducer in the x-direction. Starting at one end of the gel, B-mode videos (at least 2 seconds long) were taken approximately every 5 mm in yz-plane. Videos were saved in audio video interleave (AVI) format and exported for image analysis in MATLAB.

### Ultrasound Data Analysis

A script was written in MATLAB to take as input an AVI format video and to calculate the average brightness per pixel in each frame. To do this, the script prompted the user to select a region of interest in the gel, and average brightness in that region was calculated in each of the frames of the video. Thus the script output an array of brightness values for each frame, essentially calculating the change in brightness over time in the region of interest. A blank gel was used to determine background levels of brightness, and the background was subtracted out from the brightness values. The result was an array for each video showing the change in contrast over time provided by the microbubbles in the region of interest, or in other words, the effect of ultrasound insonation on the mechanical stability of microbubbles.

### Results

### Dissolution Studies

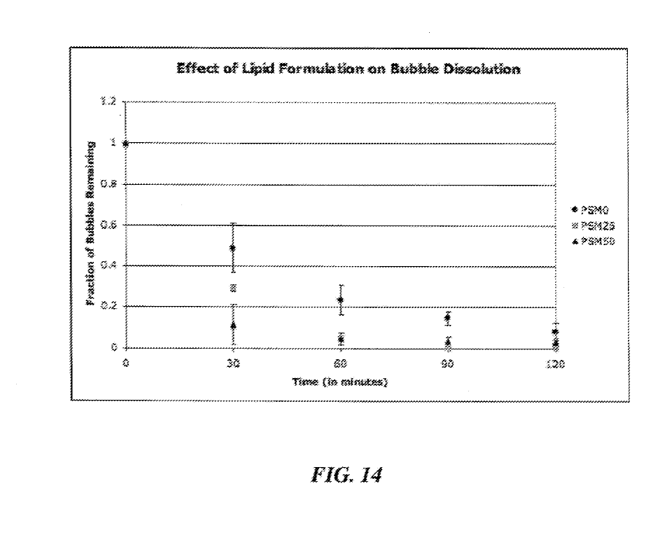

Microbubbles were produced from all lipid formulations and 3 samples of each formulation were analyzed for dissolution in a salt solution (Isoton). The results of this experiment are shown in FIG. 14. Dissolution was observed for microbubbles formed from various lipid formulations in a salt solution over 2 hours, and is displayed as a fraction of microbubbles remaining in ±10% window about median at time zero.

The fastest dissolution occurred for PSM50, the lipid formulation with the highest amount of diacetylenic lipids, while the most enduring microbubbles in the salt solution were those absent of diacetylenic lipids. At 30 minutes the difference between PSMO and PSM50 was large (49% versus 10%) and statistically significant (p=0.015), though by 2 hours the remaining proportion for all formulations was under 10%.

### Ultrasound Characterization of Microbubbles

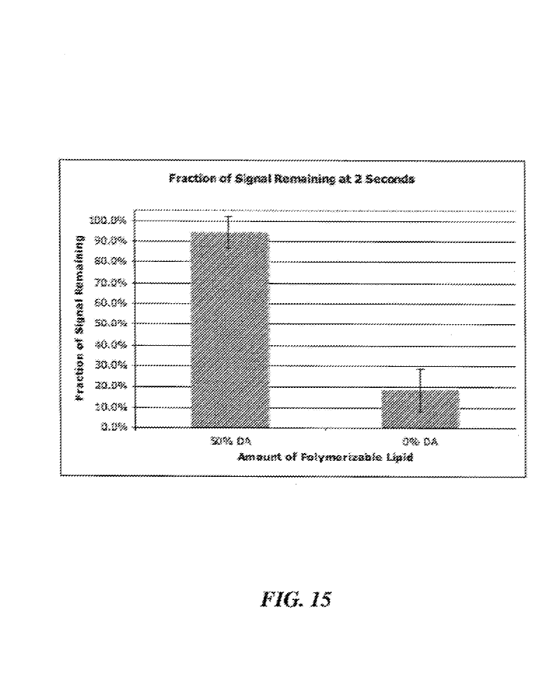

Microbubbles were successfully imaged using the Terazon ultrasound system, and a 50% polymerized lipid sample (UV treated for 5 minutes) was compared to a standard lipid formulation. Likewise, an empty gel used to quantify background brightness. Brightness was quantified in MATLAB as described previously. FIG. 15 shows the amount of ultrasound contrast (signal above background) remaining after 2 seconds of ultrasound insonation, normalized to the starting brightness. For the standard lipid formulation 18.4% (SD 10.3%, n=3) of the original contrast remained, while the 50% polymerized lipid microbubbles had 94.5% (SD 7.6%, n=8) of the contrast remaining after 2 seconds. Using a t-test to compare the means, the null hypothesis returned a p-value of 0.0017, indicating a statistically significant difference.

The graph shows the amount of ultrasound contrast remaining after 2 seconds for microbubbles with 50% polymerized lipids (PSM50, 50% DA) and those with a standard formulation (PSMO, 0% DA).

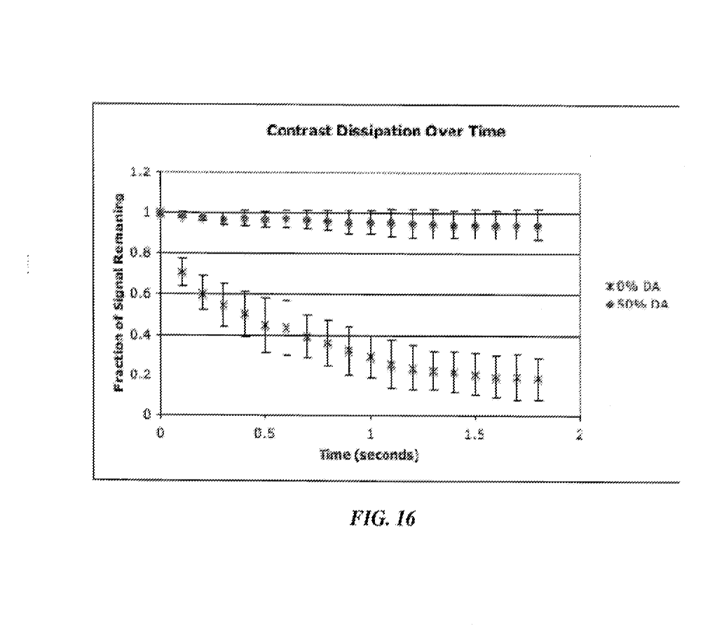

In order to fully understand these results, it was important to look at the time dependence of the microbubble destruction. Using the same data previously presented, FIG. 16 shows the change in ultrasound contrast over the 2 seconds of ultrasound insonation for the two samples. Most of the ultrasound contrast for the standard formulation was lost in the first second, with the loss of contrast beginning to taper off. The polymerized lipid microbubbles appear to hold relatively constant, losing a small amount of contrast progressively over the 2 seconds.

### Discussion

The purpose of this example was to investigate the effects of the polymerized shell on microbubble properties, and to characterize properties of interest for applications in ultrasound contrast imaging. The ultrasound imaging studies demonstrated the echogenicity of the lipid microbubble system, both for polymerized lipid and standard lipid formulations. This result confirmed the feasibility of the system for ultrasound contrast applications. Additionally, the ultrasound studies demonstrated the increased stability of PSMs under ultrasound insonation relative to standard lipid microbubbles. Following 2 seconds of ultrasound insonation, PSMs still provided 94.5% of the initial contrast level, while the contrast provided by the standard microbubbles decreased to 18.4%.

Microbubbles are excellent ultrasound contrast agents due to the large difference in acoustic impedance between the gas-filled core and the surrounding environment, which results in high levels of acoustic reflectance. Depending on the frequency of ultrasound waves, the microbubbles oscillate either symmetrically or asymmetrically, and can be destroyed by mechanical force at high sound intensities. By crosslinking the lipid components that form the microbubble shell, this example shows that one can increase the stability of the microbubbles in ultrasound, offering greater mechanical stability to help counter microbubble destruction.

Microbubble dissolution is highly relevant to ultrasound contrast applications because it determines circulation time. For ultrasound molecular imaging, it is important to have microbubbles that will last long enough to circulate through the system and bind their target, but that will also dissolve on a short time scale after the imaging session.

To optimize microbubble circulation time, it is important to address both microbubble aggregation and coalescence and microbubble collapse and dissolution. The mechanical stability provided by polymerized lipids, evidenced by the ultrasound studies, offers a mechanism for preventing microbubble collapse and dissolution. Crosslinking of the lipid constituents allows for greater mechanical integrity, making the shell more mechanically and, theoretically, chemically resistant. The addition of lipids with grafted PEG has already been shown to increase stability of microbubbles in solution (Talu et al. 2006, Langmuir 22, 9487-9490), though optimization of mole fractions and PEG length has yet to be done. Surface charge on lipid microbubbles has been shown to be related to capillary retention time in vivo (Fisher et al. 2002, J. Am. Coll. Cardiol. 40, 811-819). By taking advantage of these variables, further optimization of microbubble properties may be possible in future works.

### Example 3

### Drug Encapsulation of Microbubbles

This example demonstrates the drug encapsulation capabilities of the microbubble system. Microbubbles were conjugated with targeting molecules through a lipid tether and specific binding confirmed.

Drug delivery potential was demonstrated through encapsulation of a fluorescent dye in the microbubbles followed by fluorescent microscopy. Additionally, microbubbles were conjugated to a protein through a biotin-avidin system and conjugation likewise confirmed using fluorescent microscopy.

### Design and Methods

This example confirms the hypothesis that microbubbles can be used to encapsulate molecules of interest, and that proteins of interest can be conjugated to microbubbles through a polymeric tether.

### Drug Encapsulation

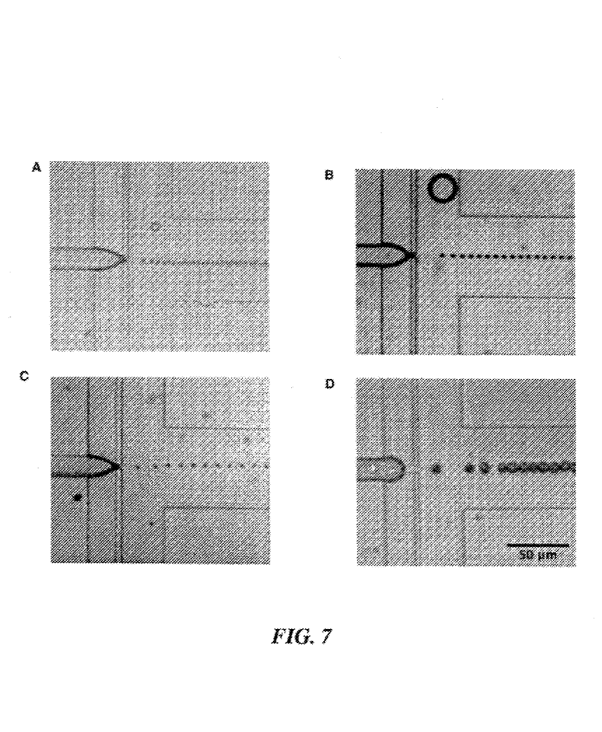

In order to demonstrate the potential of the microbubbles as drug delivery agents, a lipophilic dye was encapsulated in the lipid monolayer and imaged using fluorescent microscopy. The lipophilic dye used was nile red (7-diethylamino-3,4-benzophenoxazine-2-one). The structure of nile red is shown in FIG. 6. This lipophilic dye was used as a proof of concept for drug encapsulation studies.

It is clear from the structure of nile red that it is highly hydrophobic, like many drugs of interest such as paclitaxel. The hydrophobicity of the dye forces it to localize in the lipid monolayer rather than remain in aqueous solution. Other methods of drug encapsulation are possible, such as covalent attachment, but this method was used as a proof of concept because of its simplicity.

In this experiment, nile red was encapsulated through bath sonication with a lipid solution prior to formation of the microbubbles, and fluorescent microscopy was used to qualify the distribution of nile red in the resulting microbubble solution. Likewise, a negative control of microbubbles without nile red was visualized. To this end, 0.1 mg of nile red (Sigma, St. Louis, Mo.) was added to 1 mL of non-polymerizable lipid solution and subjected to bath sonication for 30 minutes. A negative control of 1 mL lipid solution without nile red was also prepared. Following sonication, the lipid solution was used to make microbubbles as described previously. Following microbubble production, microbubble solutions were sandwiched between glass cover slips and observed under brightfield and fluorescent microscopy (rhodamine filter, 2 second exposure time) using a Zeiss Axiovert S100 microscope (Zeiss, Oberkochen, Germany).

### Conjugation Protocol

In order to demonstrate conjugation of a protein to the microbubbles through a lipid tether, a biotin-avidin system was used. This proof-of-concept study used biotinylated lipids to attach fluorescently tagged avidin protein. The resulting structure was visualized using fluorescent microscopy and compared to a negative control absent of biotinylated lipids.

### Fluorophore-Protein Attachment

In order to visualize the conjugated protein it was necessary to covalently attach a fluorescent dye through a chemical coupling reaction prior to conjugation. To this end, 0.5 mg of NeutraAvidin (Thermo Fisher, Waltham, Mass.) was diluted in 1 mL of phosphate buffered saline (PBS). A 70-fold molar excess of Alexa Fluor 488 succinimidyl ester (Molecular Probes, Eugene, Oreg.) was added to the protein (mixed through inversion of Eppendorf tube) and incubated for 1 hour for amine labeling. Subsequently, the labeled protein was separated from free dye using a size-exclusion PD-10 column (Amersham Biosciences, Piscataway, N.J.). The column protocol was as follows: (i) Equilibrate column with 20 mL PBS, (ii) Add 1 mL of protein solution to the column (allow full entry), (iii) Add 2 mL of PBS to the column (allow full entry), and (iv) Add 1 mL PBS to the column and collect the eluted protein (twice).

The resulting fluorescently tagged protein solution was stored at −20° C., and thawed prior to the subsequent conjugation steps.

### Microbubble Conjugation and Visualization

The lipid formulations used in this experiment were PSMO and a modified PSMO formulation containing 5% PE-PEG2000-biotin. The PSMO formulation was used as a negative control since only non-specific binding should occur, as no biotin was present to bind the protein. Microbubbles were produced using the microfluidic method and dispersed 1 to 10 in 10:10:80 aqueous solution. The dispersed microbubble solution was then incubated with the fluorescently tagged protein solution at a ratio of 1:2 for 45 minutes. Subsequently, 200 μL of the incubate was sandwiched between two glass coverslips and brightfield and fluorescent images (FITC filter, 2 second exposure) were obtained using a Zeiss Axiovert 5100 microscope (Zeiss, Oberkochen, Germany).

### Results

### Drug Encapsulation

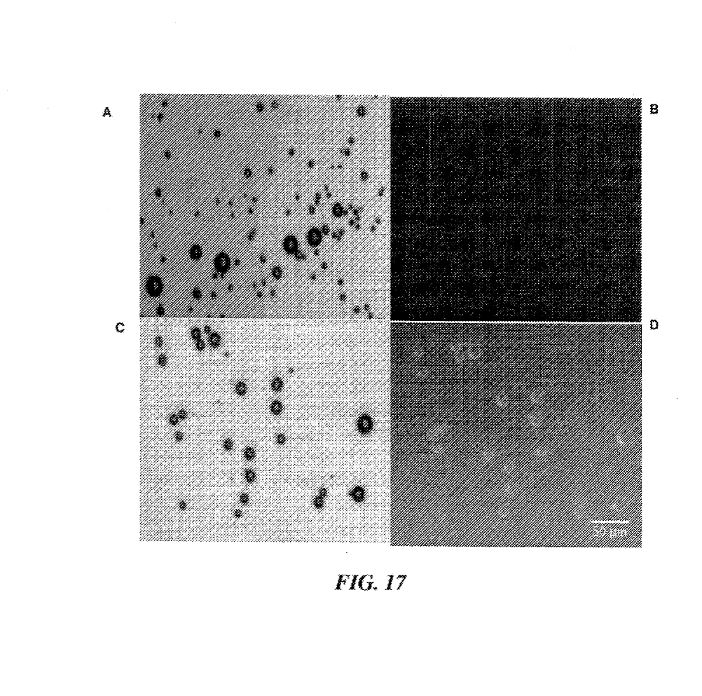

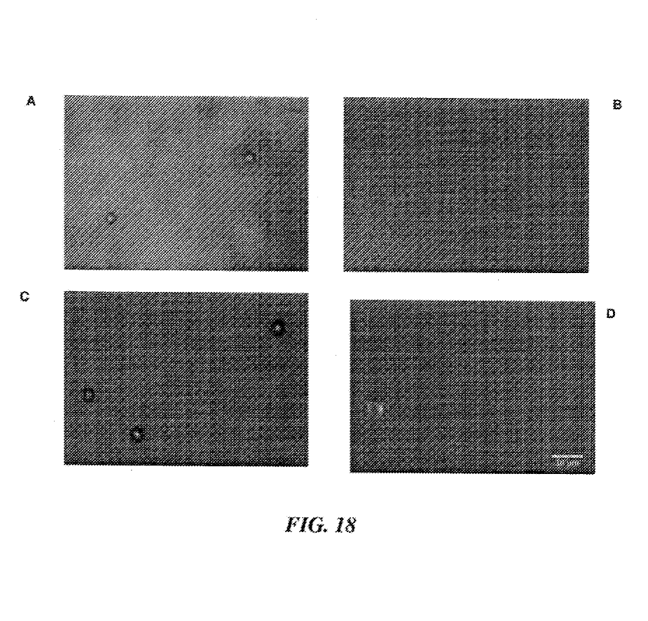

Lipids with nile red were successfully prepared as described. The lipid solution turned red as the nile red powder was dispersed throughout the solution during the bath sonication. A variety of microbubble sizes were produced, as it was unclear at what volume the encapsulated dye would be successfully visualized using our system. Fluorescent and brightfield microscopy images were obtained of both the nile red encapsulated sample and the negative control. These images are shown in FIG. 17.

From these images it can be seen that when no nile red was encapsulated through the sonication method, there was no fluorescence from the microbubbles (FIG. 17, A and B), as would be expected for standard lipid microbubbles. However, when nile red was encapsulated, the microbubbles did fluoresce under the rhodamine filter, as would be expected for encapsulated nile red (FIG. 17, C and D).

### Conjugation Studies

In the conjugation studies, biotinylated microbubbles and non-biotinylated microbubbles were incubated with fluorescently tagged NeutrAvidin. The results of this experiment are shown in FIG. 18. Qualitatively, biotinylated microbubbles showed far greater binding of the fluorescent NeutrAvidin than non-biotinylated microbubbles. This was demonstrated by the localization of fluorescence around microbubbles in FIG. 18d and the absence of localization in FIG. 18b. This experiment did suffer from low microbubble retention, with relatively few microbubbles remaining after the incubation step in PBS.

Microbubbles were incubated with fluorescent NeutrAvidin protein. (A) Brightfield image of PSMO microbubbles with no biotinylated lipids. (B) Fluorescent image of same field as A (FITC, 2 second exposure). (C) Brightfield image of microbubbles containing biotinylated lipids. (D) Fluorescent image of C (FITC, 2 second exposure). Scale bar is for all images.

### Discussion

The goal of this example was to demonstrate, the feasibility of our system for drug delivery and targeted imaging applications. To this end, the encapsulation of a hydrophobic dye in the lipid monolayer successfully showed the potential of the microbubbles to carry a payload. The fluorescent microscopy images clearly showed retention of nile red in the lipid shell, opening the door for further encapsulation studies with more clinically relevant molecules.

The purpose of the conjugation studies was simply to show the specific attachment of a protein to the microbubbles through a lipid tether. A biotin-avidin system was used due to its simplicity and ubiquity. These studies did show protein attachment, with fluorescent microscopy showing localization of Alexa Fluor 488 tagged NeutrAvidin around biotinylated microbubbles. This experiment, though only a first step toward microbubble targeting in vivo, was an important hurdle to overcome prior to in vitro binding studies.

This example successfully demonstrated the feasibility of our system for drug delivery and imaging applications. By following similar procedures clinically relevant drugs can be encapsulated and pathologically relevant antibodies or other targeting agents can be conjugated to the microbubbles.

### Example 4

### Tuning of Microbubble Stability Under Ultrasound

This example demonstrates that the acoustic stability of polymerized shell microbubbles under ultrasound (7.5 MHz) was tunable by varying the amount of diacetylene lipid in the microbubbles. Monodisperse microbubbles, composed of photopolymerizable diacetylene lipids and phospholipids, were produced by microfluidic flow focusing. The stability of the polymerized shell microbubbles in the bubble suspension and acoustic stability under ultrasound field were significantly greater than for nonpolymerizable shell microbubbles and commercially available microbubbles (Vevo MicroMarker). Polymerized microbubbles containing higher diacetylene lipid content showed less dissolution under ultrasound than lower diacetylene content. VMM or the nonpolymerizable formulations showed fast decrease of ultrasound image brightness, indicating rapid microbubble destruction.

### Design and Methods

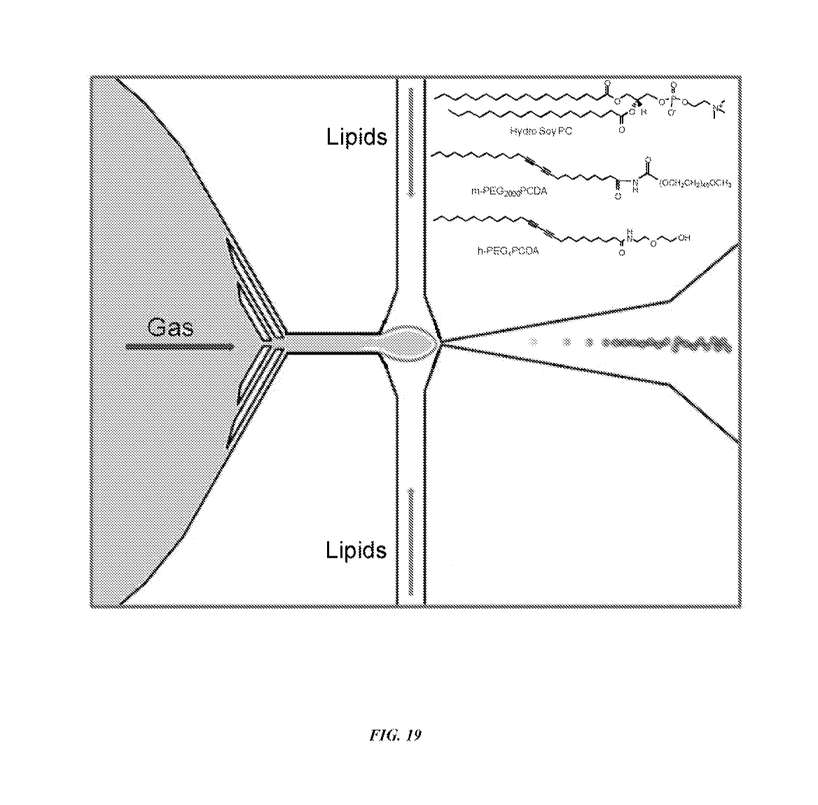

The microfluidic flow focusing device design (FIG. 19) was modified from the microfluidic flow focusing device described by Hettiarachchi et al. (Hettiarachchi et al. 2007, Lap Chip 7, 463-468). Gas enters the device through the central 40 μm channel, and is focused through the orifice (6 μm) by an aqueous lipid mixture dispersion, which flows through the flanking 50 μm channels. This focusing of the flow results in a microjet which breaks at the orifice into microbubbles with the formation of a monolayer of lipids at the gas-water interface. Photolithography techniques were used for fabrication of the poly(dimethylsiloxane) (PDMS)-based microfluidic flow-focusing device using a chrome photomask and a silicon wafer (SI-Tech, Inc., Topsfield, Mass.). The wafer was spin-coated with SU8-2005 (MicroChem, Newton, Mass.) to a thickness of 5 μm, followed by baking and developing of the photoresist layer. The wafer was used as a mold for PDMS (Sylgard 184, Dow Corning, Midland, Mich.) devices. PDMS microfluidic devices, hole-punched for portholes, were plasma-treated in an ML4 Plasma Asher (PVA TePla, Corona, Calif.) to bond to glass cover slips. The final devices were plasma-treated again (Harrick PDC-32G Plasma Cleaner, Ithaca, N.Y.) for 5 min before use to render the surfaces hydrophilic to facilitate complete wetting of the interior of the devices.

For a polymerizable lipid mixture, ethylene glycol diacetylene lipids (h-PEG₁PCDA), PEG2000-diacetylene lipids (m-PEG₂₀₀₀PCDA) (NanoValent Pharmaceuticals, Inc., Bozeman, Mont.) and L-α-phosphatidylcholine, hydrogenated (Soy) (hydro soy PC) (Avanti Polar Lipids, Alabaster, Ala.) (FIG. 19) were used varying h-PEG₁PCDA from 0 to 15 mol % and keeping m-PEG₂₀₀₀PCDA constant at 15 mol %, with the balance consisting of hydro soy PC. The mixture of lipids in chloroform was evaporated in vacuum and the dry film was hydrated with a 5/5/90 (v/v/v) solution, which consists of 5% glycerin, 5% propylene glycol (Sigma-Aldrich, St. Louis, Mo.), and 90% water, resulting in a total concentration of the lipid of 5.32 μmol/mL. The mixture was stirred at 67° C. for 1.5 h and bath sonicated at 67° C. for 3 hr or more until the dispersion became clear. For a nonpolymerizable lipid shell formulation, 15 mol % of 1,2-distearoyl-sn-phosphoethanolamine-PEG2000 (m-PEG₂₀₀₀-DSPE) (Avanti Polar Lipids) and 85 mol % of DSPC were used, followed by the same preparation method for the mixture dispersion.

A 0.05% (v/v) Tween 20 (Sigma-Aldrich) solution was pre-run in the microfluidic device to create a predictable microchannel surface and to prevent microbubbles from sticking to the microchannel wall or from clogging the orifice. The lipid dispersion (kept at 80° C.) was pumped into the device using a digitally controlled syringe pump (Harvard Apparatus PHD2000, Holliston, Mass.) at a constant flow rate from 2.0 to 3.0 μL/min Decafluorobutane gas (Synquest Laboratories, Alachua, Fla.) was injected to the device from the gas tank attached to a pressure regulator (Swagelok, Solon, Ohio), followed by a needle valve and a pressure meter. As the microbubbles were produced, they were polymerized under UV light at 254 nm (8W model, UVP, LLC., Upland, Calif.). Microbubble polymerization was determined by observing the color of the bubbles, which turns from clear to blue, purple, or red, depending on the exposure time or cross-linking density (Lifshitz et al. 2009, Langmuir 25, 4469-4477).

Microbubble production was monitored using a phase contrast microscope (Axiovert 25, Zeiss, Oberkochen, Germany), and images were captured with a high-speed camera (Photron, San Diego, Calif.). When the solution containing microbubbles exited the output port onto the PDMS device, the solution was covered with a glass slide for imaging. Histograms of microbubble size were obtained using Image J software. The polydispersity, σ=δ/d_{avg}×100%, was also calculated from the average bubble size d_{avg} and standard deviation δ. Microbubbles immobilized in a polyacrylamide (PAAM) gel were visualized using a portable diagnostic ultrasound system (Terason 2000, Teratech, Burlington, Mass.) along with a 5-10 MHz clinical ultrasound transducer (L10-5, Terason). Ultrasound images were taken every 10 sec for 2 min, every 20 sec for 3 min, and every 1 min for 10 min Ultrasound echogenicities of each microbubble type at the selected area were analyzed by brightness intensity values measured using a function called Z-project in Image J. where the brightness intensity is based on the percentage of the total number of pixel values from 0 to 255.

### Results

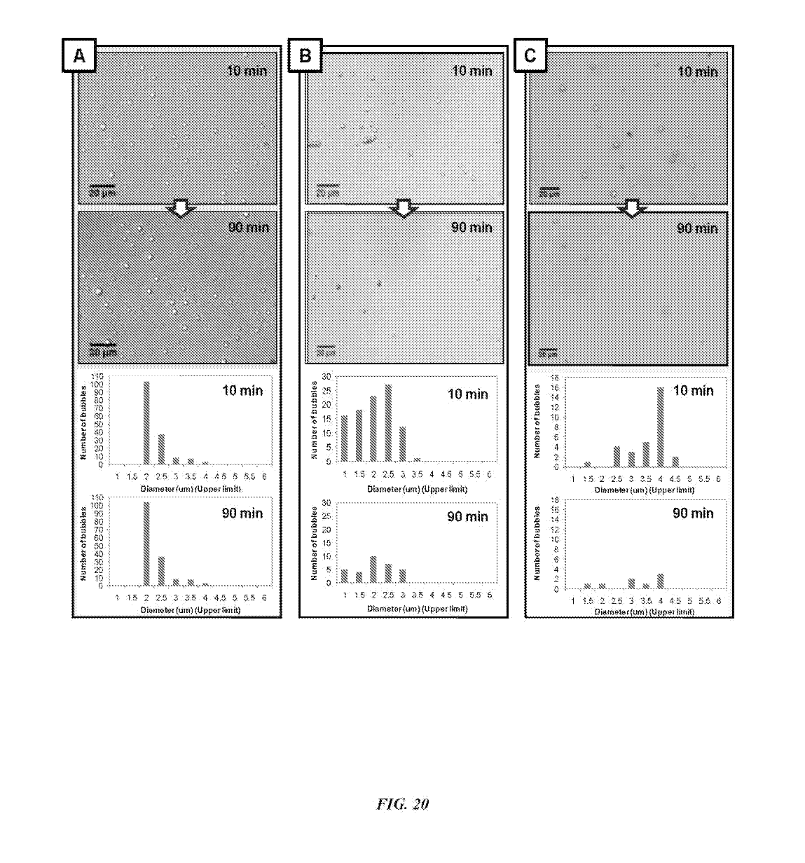

To compare the robustness of the different microbubbles, the lifetime of the bubbles were observed 10 min after formation and then again after 90 min. It was found that approximately one third of the Vevo MicroMarker (VMM) (FIG. 20A) remained intact after 90 min, and the size of the bubbles decreased from d_{avg}=4.7 to 3.0 μm, presumably due to dissolution of the larger bubbles. The standard deviation (6) of the VMM was 1.0 at 10 min. The polydispersity of the nonpolymerizable shell microbubbles (NSM) produced by the microfluidic focusing device was, initially 4.5%, at 10 min, which corresponds to 6=0.14 (FIG. 20B). However, the polydispersity increased to 47% at 90 min, and most of the bubbles decreased in size from d_{avg}=3.1 to 1.6 μm or disappeared. The polymerized shell microbubbles (PSM) containing 30 mol % of polymerizable diacetylene lipids (30% DA: 15 mol % h-PEG₁PCDA and 15 mol % m-PEG₂₀₀₀PCDA), showed almost the same values in number, size, and distribution, at 10 and 90 min (FIG. 20C). The average diameters at 10 and 90 min were 3.0 and 2.9 μm, respectively, and the standard deviations of the bubbles were 0.62 and 0.60, which are less than that of VMM. About 50% of the PSM remained intact even after 15 hr (data not shown). These results indicate the dissolution rate of the PSM is significantly slower than VMM or NSM. Aggregation of the microbubbles for VMM was observed, but the aggregated microbubbles can be redispersed under slow flow conditions. Coalescence or fusion were not observed from any of the microbubbles that were tested.

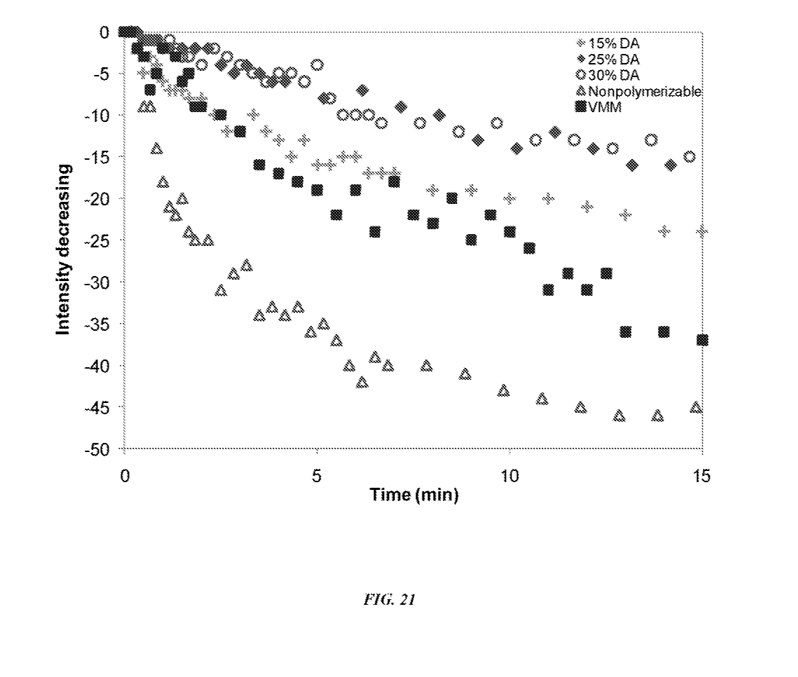

Time-intensity curves, corresponding to ultrasound echogenicity, were obtained at 7.5 MHz for NSM, VMM, and polymerized shell microbubbles containing 15, 25, and 30 mol %, called 15% DA, 25% DA, and 30% DA, respectively (FIG. 21). The data represent an average of two or three measurements, and were generally reproducible. The NSM showed significant decrease in brightness intensity within a few minutes, and the absolute amount decreased after 15 min was 45, which may be due to microbubble destruction. Intensities of the VMM decreased gradually and the final value at 15 min was −37. The 25% DA or 30% DA showed little decrease by 15 min, which is a decrease three-fold smaller than for the NSM. These data indicate that the polymerizable lipids increased not only the stability in solution but also the stability under ultrasound, offering greater mechanical stability to help counter microbubble destruction. We observed a higher dissolution rate and a more rapid decrease in intensity for 15% DA than for 25% or 30% DA. However, even this was still a slower dissolution rate that what was measured for VMM or NSM. Importantly, this shows that we can tune the dissolution rate by controlling the amount of DA in the shell. The differences in dissolution rate as a function of concentration of DA suggests that the structure of the shell is modulated by lipid composition. There is evidence in the literature that supports the idea that the shell most likely consists of phase-separated lipids. For example, Gaboriaud et al. showed that mixed films of dimyristoylphosphatidylcholine and 10,12-tricosadiynoic acid (a homolog of PCDA) on a Langmuir surface segregated into islands of similar lipids. The determination of the degree of phase-separation of PCDA and hydro soy PC lipids in these polymerizable microbubble formulations is beyond the scope of this study, and here we can only conclude that the acoustic stability increases with increasing the polymerized area on the shell.

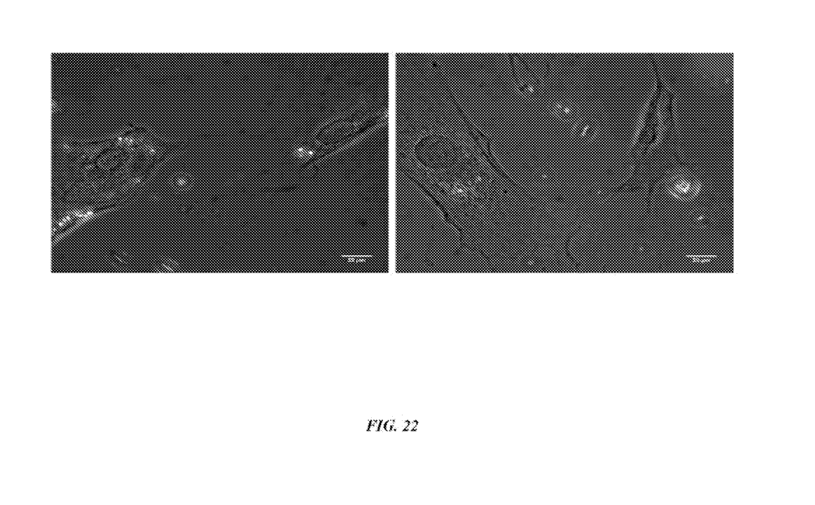

FIG. 22 shows the results of using targeted microbubbles on bovine smooth muscle cells. Bovine vascular smooth muscle cells were cultured onto glass coverslips. A solution containing microbubbles with or without the RGD conjugated to the surface were added to wells containing the cells cultured on the glass coverslips. Phase contrast images were taken using a Zeiss Axiovert S100 microscope. The microbubbles that had the peptide sequence RGD attached to them were able to bind to cells. FIG. 22B shows that non-targeted microbubbles do not bind to bovine smooth muscle cells.

### Discussion

Polymerized shell microbubbles (PSMs) of controlled size and distribution were produced using microfluidic focusing, which can potentially give enhanced and prolonged signal in molecular imaging of the vasculature. The PSMs remained intact much longer in an aqueous solution than nonpolymerizable shell microbubbles or commercially available microbubbles (VMM). Under ultrasound field, PSMs containing a higher diacetylene lipid content showed less dissolution under ultrasound (7.5 MHz) than lower diacetylene content, and much less than VMM or nonpolymerized formulations. These results imply that the dissolution of microbubbles in the bloodstream or under ultrasound stimulation is tunable by varying the fraction of polymerizable lipid. One can optimize ultrasound contrast agents, which can last long enough to circulate through system of interest and bind their target.

### Example 5

In this example, various methods are described to optimize microbubbles for stability against dissolution and size distribution stability. Some of the characteristics that can be modified to achieve stability and size homogeneity are PEG length, PEG mole fraction, surface charge density, polymerized lipid mole fraction, antibody tether length, and antibody tether mole fraction. These studies give a predictive ability that will reduce the need for additional empirical experimentation to optimize the microbubbles.

To test the effects of PEG length, polymerizable shell microbubbles containing 15% ethylene glycol diacetylene lipids (h-PEG₁PCDA), 15% PEGX-diacetylene lipids (m-PEG_{X}PCDA) (NanoValent Pharmaceuticals, Inc., Bozeman, Mont.) and 70% L-α-phosphatidylcholine, hydrogenated (Soy) (hydro soy PC) (Avanti Polar Lipids, Alabaster, Ala.) are used, varying the molecular weight X of the attached PEG to be 500, 1000, 2000, 3000, 4000, or 5000. The microbubbles are generated by microfluidic flow focusing and polymerized by UV as described, and stability and size of the microbubbles are both measured as described in Examples 2 and 4. Further, the effects of PEG length on the stability of drug encapsulation are tested as described in Example 3. Microbubble sensitivity to different acoustic parameters are also be tested, such as differential ultrasound stability under different ultrasound settings, which is necessary to optimize protocols for imaging at one frequency and destruction and drug release at another. These studies into ultrasound-induced drug release have direct clinical applications

Based on these results, additional tests (such as increasing PEG size to a molecular weight greater than 5000) are performed to find the optimal practical PEG to incorporate. The m-PEG_{X}PCDA with the most optimal PEG size of those tested is then be used for additional studies, to test how varying the percentage of PEGylated lipid in the PSMs affects stability, size distribution, and drug incorporation.

Additional studies examine the effects of increasing amounts of charged lipids on microbubble function. Hydro soy PC is zwitterionic, so microbubbles are generated with constant amounts of h-PEG₁PCDA, and m-PEG₂₀₀₀PCDA, while varying the percent composition of hydro soy PC from 70% to 0%, replacing the zwitterionic lipid with a lipid with an uncharged head group such as a hydroxy or methoxy. The effects of head group charge are further tested by replacing the zwitterionic lipid with a lipid containing either a positive or negatively charged head group. Stability, size, and drug incorporation are likewise measured as described in earlier examples.

Based on these results, further optimization of polymerizable lipid content is achieved by comparing microbubbles containing equal fractions of lipids by charge content, while varying only the fraction of lipids containing polymerizable diacetylenic groups. Additionally or alternatively, microbubbles with the same molar content of polymerizable lipids are treated with different intensities and durations of UV light to achieve partial to full polymerization for further testing.

Finally, microbubble fabrication and storage of produced microbubbles are similarly optimized through empirical testing. This occurs through the scaling up of the microfluidic system or through similar but distinct methods, such as forcing gas through a porous thin film into a lipid solution. Likewise, studies also include investigations into the effects of lyophilization or centrifugation of microbubbles. Also, the microbubbles in suspension are separated from the liposomes that were produced as a side-product of the microfluidic focusing technique described in example 1, using the vast differences in properties between the gas-filled 5 μm microbubbles and the liquid-filled 200 nm liposomes, such as through size differentiation or by allowing the gas-filled microbubbles to float to the surface of the suspension. These optimization steps allow the microbubbles to be efficiently used for both diagnostic and therapeutic clinical applications.

### Example 6

For drug delivery applications, microbubbles are optimized for encapsulating not just Nile red, as described in Example 3, but also paclitaxel, one example of a drug of clinical interest. In addition to the variables optimized in Example 5, more efficient drug encapsulation is achieved by increasing drug encapsulation volume or utilizing other methods of encapsulation. Drug encapsulation volume may be increased by adding oil into the emulsion system to increase the volume of the hydrophobic shell. Drugs may also be incorporated through covalent attachment of drug-carrying polymer nanoparticles through a PEG tether, which increases the total surface area to which the drugs may be attached.

Efficiency of drug encapsulation is measured by conjugating a fluorescent label to the drug, such as fluorescein, which fluoresces in a wavelength range different from the polymerized microbubbles. Microbubbles containing the drug are produced through microfluidic flow focusing a lipid solution mixed with the labeled drug, and any unincorporated drug is removed from the surrounding solution by washing the microbubbles. The resulting microbubbles are measured through flow cytometry to determine the fluorescence intensity distribution, which will reflect both the efficiency and consistency of drug incorporation.

### Example 7

For targeted molecular imaging and drug delivery, the conjugation protocol described in Example 3 could be modified and optimized. To avoid an immunogenic response to the microbubbles, the biotin-avidin could be replaced with a covalent attachment method such as through maleimide or disulfide chemistry. Likewise, optimization of tether length and mole fraction of lipids containing the tether allows for improved conjugation and binding efficiencies. Tethering is useful for both incorporating drugs covalently to the lipid shell and for attaching targeting agents. In this example, anti-IgG can be used as the tethered targeting agent, but any targeting or therapeutic agent may be used. PSMs are conjugated by maleimide chemistry to anti-human IgG. Binding is measured in vitro using a flow chamber coated with human IgG to simulate an atherosclerotic plaque. The tethered microbubbles are then applied to the flow chamber at flow rates mimicking blood flow rates of different vessels in vivo. Phase-contrast microscopy and ultrasound are then used to observe the amount of PSMs that bind to the chamber walls at each flow rate. The type of covalent attachment, the fraction of tethers, and the types of tethered ligand on the microbubbles are also varied for additional in vitro testing and optimization, and different targets may be used in the flow chamber to test for feasibility of microbubble targeting and binding.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A microbubble comprising a polymerized lipid shell and a gas, wherein the gas is encased within the shell, and wherein the polymerized lipid shell comprises at least about 5% polymerizable lipid.

2. (canceled)

3. The microbubble of claim 1, wherein the gas is a perfluorocarbon.

4. The microbubble of claim 3, wherein the perfluorocarbon is decafluorobutane.

5. The microbubble of claim 1, wherein the gas is a mixture of at least two perfluorocarbons.

6. The microbubble of claim 1, wherein the polymerized lipid shell comprises at least one polymerizable lipid and at least one non-polymerizable lipid and has a percentage of about 5-50% polymerizable lipid.

7. The microbubble of claim 6, wherein the at least one non-polymerizable lipid is L-α-phosphatidylcholine), PE-PEG2000 (1,2-distearoyl-sn-glycero-3-phosphoethanolamine-N-[methoxy(polyethylene glycol)-2000 or PE-PEG2000-biotin.

8. The microbubble of claim 6, wherein the at least one polymerizable lipid is a diacetylenic lipid.

9. The microbubble of claim 6, wherein the microbubble is UV treated for about 2-5 minutes after fabrication to polymerize the lipid shell.

10. (canceled)

11. The microbubble of claim 1 wherein the microbubble was prepared by a microfluidic flow focusing device.

12. The microbubble of claim 1 wherein the microbubble comprises a targeting agent.

13. (canceled)

14. (canceled)

15. (canceled)

16. The microbubble of claim 1, wherein the microbubble is an ultrasound contrast agent further comprising an acceptable carrier for administration to an individual.

17. (canceled)

18. (canceled)

19. A collection of microbubbles comprising gas-filled polymerized shell lipid microbubbles, wherein the microbubbles in the collection are monodispersed and are within 2-5 μm size range.

20. (canceled)

21. The collection of claim 19, wherein the microbubbles in the collection comprise at least about 5% polymerizable lipid.

22. (canceled)

23. A method of treating an individual comprising:
administering a microbubble to an individual in need of thereof, said microbubble comprising a polymerized lipid shell, a gas, a targeting agent and a therapeutic agent, wherein the gas is encased within the shell, and wherein the polymerized lipid shell comprises at least about 5% polymerizable lipid.

24. (canceled)

25. (canceled)

26. (canceled)

27. (canceled)

28. (canceled)

29. (canceled)

30. (canceled)

31. (canceled)

32. (canceled)

33. A method of making a microbubble of claim 1 comprising:
(a) microfluidic flow focusing a mixture of polymerizable lipid and standard non-polymerizable lipid and a gas through an aperture to form micrometer microbubbles and (b) UV treating the microbubbles of step a for at least 2 minutes to polymerize the polymerizable lipid.

34. (canceled)

35. (canceled)

36. (canceled)

37. (canceled)

38. The method of claim 33, wherein the mixture of lipids comprises at least one polymerizable lipid and at least one non-polymerizable lipid.

39. The method of claim 33, wherein the polymerizable lipid makes up a percentage of about 5-50% of total lipid of the mixture.

40. The method of claim 33, wherein the non-polymerizable lipid is L-α-phosphatidylcholine), PE-PEG2000 (1,2-distearoyl-sn-glycero-3-phosphoethanolatnine-N-[methoxy(polyethylene glycol)-2000 or PE-PEG2000-biotin.

41. The method of claim 39, wherein the polymerizable lipid is a diacetylenic lipid.

42. (canceled)

43. (canceled)

44. (canceled)

45. The method of claim 41 wherein the microbubbles are treated with UV for at least 30 minutes to polymerize the polymerizable lipid.

46. (canceled)
